(19) United States
(12) Patent Application Publication
(10) Pub. No.: US 2024/0123081 A1
(43) Pub. Date: Apr. 18, 2024

(54) BRANCHED MOIETY FOR USE IN CONJUGATES

(71) Applicant: MEDIMMUNE, LLC, GAITHERSBURG, MD (US)

(72) Inventors: Nazzareno DIMASI, Gaithersburg, MD (US); Amit KUMAR, Gaithersburg, MD (US)

(21) Appl. No.: 17/769,041

(22) PCT Filed: Oct. 25, 2019
(86) PCT No.: PCT/US2019/058120

§ 371 Date: Apr. 14, 2022

(51) Int. Cl. A61K 47/68; A61K 47/54; C07D 207/452

(52) U.S. Cl. CPC A61K 47/6889; A61K 47/545; A61K 47/6803; A61K 47/68031; A61K 47/68033; A61K 47/68035; A61K 47/68037; A61K 47/6831; A61K 47/6851; C07D 207/452

(57) ABSTRACT

A tri-functional linker moiety: formula (I), where X and Y are linking chains, and its use for preparing dual-mechanistic drug conjugates, preferably in a site-specific manner.

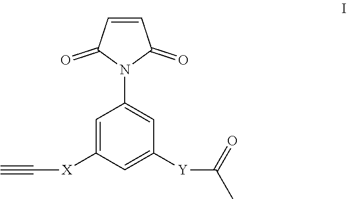

The present invention relates to branched moieties and their use in conjugates,

### BACKGROUND TO THE INVENTION

Antibody-drug conjugates (ADCs) combine the target specificity of a monoclonal antibody with the potent cell-killing activity of cytotoxic warheads. FDA approval of Kadcyla and Adcetris has given impetus to designing new ADC formats with a variety of chemotherapeutics demonstrating distinct mechanisms of action currently undergoing clinical development for hematological cancers and solid tumors (Beck 2014). The majority of ADCs, either approved or under clinical development utilize classical conjugation methods that produce heterogeneous conjugates, thereby complicating analytical characterization and batch reproducibility. Native cysteine-maleimide conjugation faces an additional problem of serum stability (Junutula 2008). The hurdles faced by the traditional biorthogonal methods can largely be addressed by utilizing site-specific conjugation. This strategy permits a precise control of the drug conjugation thereby eliminating the complication of analytical characterization and batch reproducibility (Agarwal 2015, Behrens 2014, Panowski 2014).

Site specific conjugation is an effective tool for generating ADCs with a single chemotherapeutic agent. However, ADCs with single chemotherapeutic agents are ineffective in treating heterogeneous tumors as heterogeneous cell populations have differential drug sensitivities. A potential way to overcome these obstructions is by codelivery of two or more therapeutic agents (Agarwal 2015, Behrens 2014, Panowski 2014, Fanale 2014, Younes 2013, Stern 2015, Loganzo 2015).

Codelivery of therapeutic agents with different anticancer mechanisms can overcome drug resistance as well as generate synergistic anticancer effects that may reduce individual drug-related toxicity by enhancing the antitumor efficacy (Tang 2016).

Codelivery of ADCs in combination with unconjugated, clinically approved anticancer drugs has already been attempted (Fanale 2014, Younes 2013). Moreover, it has been shown that, both in the clinical and preclinical environment, drug resistance to a particular ADC can be overcome by changing the warhead of the ADC (Stern 2015, Loganzo 2015). For reasons detailed above, an ADC carrying two mechanistically different payloads may prove instrumental towards overcoming drug resistance, However, only a few examples of heterotrifunctional linkers capable of providing a flexible platform for site specific assembly of dual drug ADCs exist in the literature (Tang 2016, Li 2015). Tang 2017 has reported the preparation of random lysine-linked dual drug ADCs carrying the cytotoxins, DM1 (N2′-deacetyl-N2′-(3-mercapto-1-oxopropyl)-maytansine) and MMAE (monomethyl auristain E). Levengood 2017 details the preparation of an ADC carrying two classes of auristatin payloads conjugated to the hinge disulfides via classical native cysteine-maleimide conjugation.

### SUMMARY OF THE INVENTION

In a general aspect the present invention provides the tri-functional linker moiety:

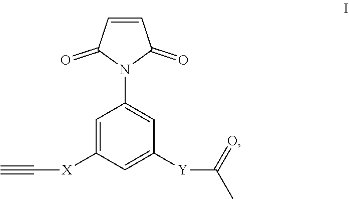

where X and Y are linking chains, and its use for preparing dual-mechanistic drug conjugates, preferably in a site-specific manner.

A first aspect of the present invention comprises a compound with the formula I:

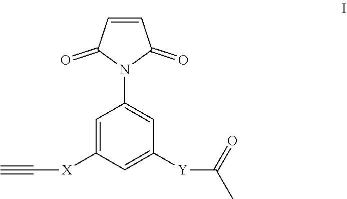

where:
X is —(C(═O)—NH)ₓₐ—(CH₂)_{xb}—(C₂H₄O)_{xc}—(CH₂)_{xd}—^{≡}, where xa is 0 or 1, xb is 0-3, xc is 0 to 4 and xd is 0-3; and
Y is —(C(═O)—NH)_{ya}—(CH₂)_{yb}—(C₂H₄O)_{yc}—(CH₂)_{yd}—(NH—C(═O))_{ye}—(CH₂)_{yf}—^{C═OMe}, where ya is 0 or 1, yb is 0-3, yc is 0 to 4, yd is 0-3, ye is 0 or 1, and yf is 0-3,
with the provisos that when xc is not 0, xd cannot be 0 and when yc and ye are both not 0, yd cannot be 0.

The phenyl maleimide group may be used for attaching the heterofunctional linker to the cell binding agent via the thiol maleimide reaction in a site-specific manner; the alkyne and the keto groups of the linker 1 can be utilized for attaching two different drugs. The alkyne group can accommodate any azido decorated drug via copper-catalyzed azide-alkyne cycloaddition (CuAAC) while the ketone group can accommodate an aminooxy carrying drug via an oxime linkage.

A second aspect of the present invention provides a linker between one or two payloads and a cell binding agent comprising a moiety derived from a compound of the first aspect of the invention. Thus, the linker may comprise one of the following moieties (IIa-1, IIa-2, IIb, IIc-1, IIc-2):

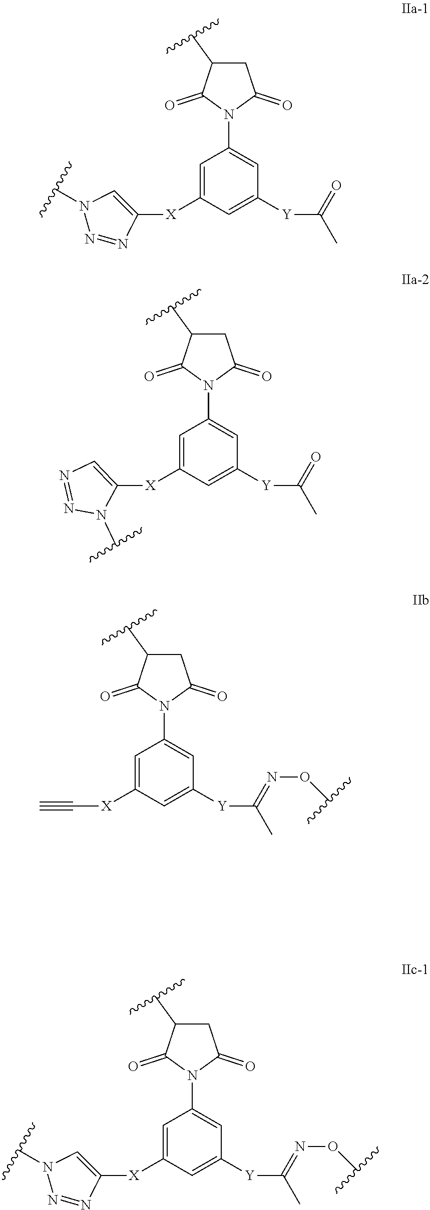

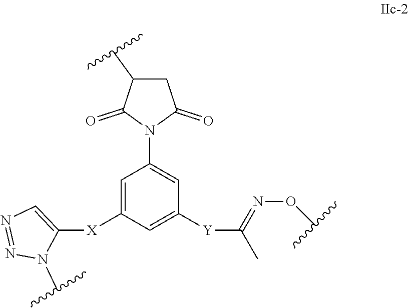

where X and Y are as defined in tne first aspect of the invention.

A third aspect of the present invention provides a conjugate of one or two payloads to a cell binding agent, wherein the linker between the one or two payloads and the cell binding agent comprises a moiety derived from a compound of the first aspect of the invention.

Thus, the conivaate may be of one of the followina formulae (IIIa-1. IIIa-2, IIIb, IIIc-1, IIIc-2):

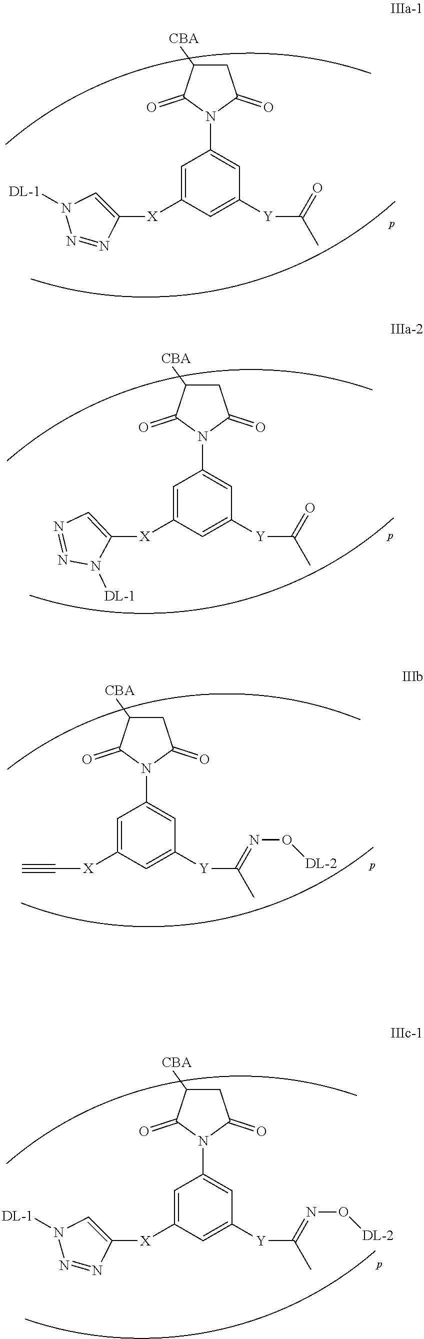

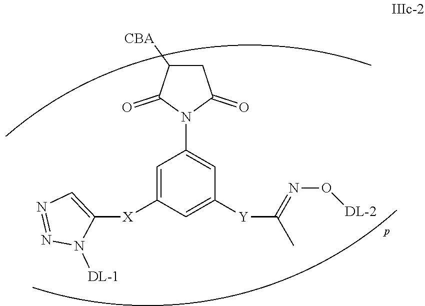

where X and Y are as defined in the first aspect of the invention, CBA is a cell binding agent, DL-1 is a first drug-linker moiety (comprising a first payload) and DL-2 is a second drug-linker moiety (comprising a second payload), and p represents the number of complete drug-linkers bound to each cell binding agent.

A fourth aspect of the present invention provides the use of a conjugate of the third aspect of the invention in the manufacture of a medicament for treating a proliferative disease. The fourth aspect also provides a conjugate of the third aspect of the invention for use in the treatment of a proliferative disease. The present invention also provides methods for the treatment of, for example, various cancers. These methods encompass the use of the Conjugates wherein Cell Binding Agent can be, for example, a protein, polypeptide or peptide, such as an antibody, an antigen-binding fragment of an antibody, or other binding agent, such as an Fc fusion protein. One of ordinary skill in the art is readily able to determine whether or not a candidate compound treats a proliferative condition for any particular cell type. For example, assays which may conveniently be used to assess the activity offered by a particular compound are described in the examples below.

A fifth aspect of the present invention provides a drug-linker comprising one or two payloads for conjugation to a cell binding agent, wherein the linker for conjugation to the cell binding agent comprises a moiety derived from a compound of the first aspect of the invention. Thus, the drug-linker may be of one of the following formulae (IVa-1, IVa-2, IVb, IVc-1, IVc-2):

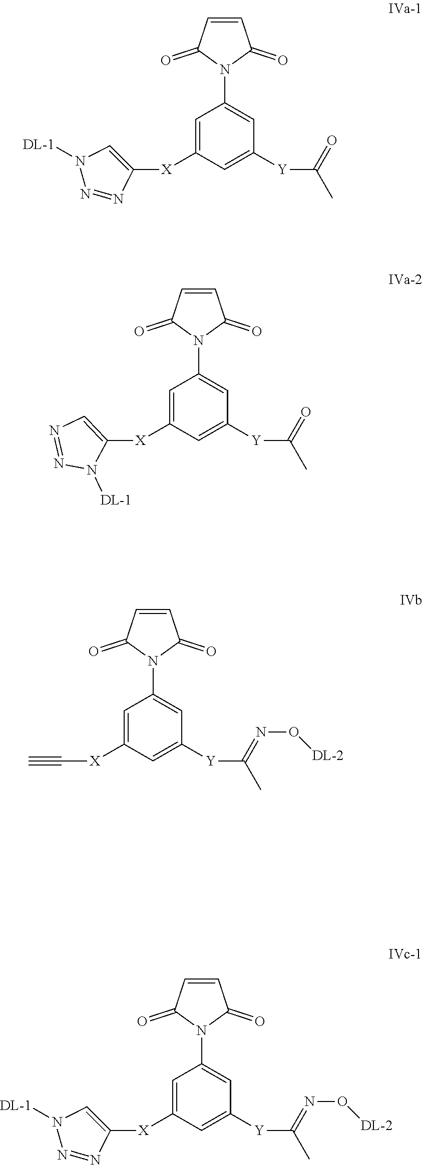

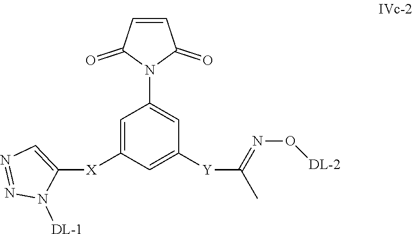

where X and Y are as defined in the first aspect of the invention, and DL-1 and DL-2 are as defined in the third aspect of the invention.

A sixth aspect of the present invention provides a modified cell binding agent comprising a moiety of formula (V):

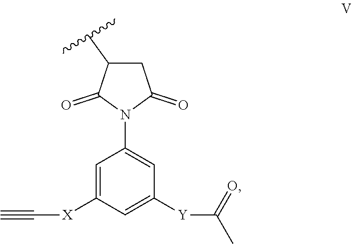

where X and Y are as defined in the first aspect of the invention.

### Definitions

### Cell Binding Agent

The Cell Binding Agent may be of any kind, and include a protein, polypeptide, peptide and a non-peptidic agent that specifically binds to a target molecule. In some embodiments, the Ligand unit may be a protein, polypeptide or peptide. In some embodiments, the Cell Binding Agent may be a cyclic polypeptide. These Ligand units can include antibodies or a fragment of an antibody that contains at least one target molecule-binding site, lymphokines, hormones, growth factors, or any other cell binding molecule or substance that can specifically bind to a target.

The terms “specifically binds” and “specific binding” refer to the binding of an antibody or other protein, polypeptide or peptide to a predetermined molecule (e.g., an antigen).

Typically, the antibody or other molecule binds with an affinity of at least about 1×10⁷ M⁻¹, and binds to the predetermined molecule with an affinity that is at least two-fold greater than its affinity for binding to a non-specific molecule (e.g., BSA, casein) other than the predetermined molecule or a closely-related molecule.

Examples of Cell Binding Agents include those agents described for use WO 2007/085930, which is incorporated herein.

In some embodiments, the Cell Binding Agent binds to an extracellular target on a cell. In some embodiments, the Cell Binding Agent may be a protein, polypeptide or peptide. In some embodiments, the Cell Binding Agent may be a cyclic polypeptide. The Cell Binding Agent also may be antibody or an antigen-binding fragment of an antibody. Thus, in one embodiment, the present invention provides an antibody-drug conjugate (ADC).

A cell binding agent may be of any kind, and include peptides and non-peptides. These can include antibodies or a fragment of an antibody that contains at least one binding site, lymphokines, hormones, hormone mimetics, vitamins, growth factors, nutrient-transport molecules, or any other cell binding molecule or substance.

### Peptides

In one embodiment, the cell binding agent is a linear or cyclic peptide comprising 4-30, preferably 6-20, contiguous amino acid residues. In this embodiment, it is preferred that one cell binding agent is linked to one monomer or dimer pyrrolobenzodiazepine compound.

In one embodiment the cell binding agent comprises a peptide that binds integrin αᵥβ₆. The peptide may be selective for αᵥβ₆ over XYS.

In one embodiment the cell binding agent comprises the A20FMDV-Cys polypeptide. The A20FMDV-Cys has the sequence: NAVPNLRGDLQVLAQKVARTC. Alternatively, a variant of the A20FMDV-Cys sequence may be used wherein one, two, three, four, five, six, seven, eight, nine or ten amino acid residues are substituted with another amino acid residue. Furthermore, the polypeptide may have the sequence NAVXXXXXXXXXXXXXXXRTC.

### Antibodies

The term “antibody” herein is used in the broadest sense and specifically covers monoclonal antibodies, polyclonal antibodies, dimers, multimers, multispecific antibodies (e.g., bispecific antibodies), multivalent antibodies and antibody fragments, so long as they exhibit the desired biological activity (Miller 2003). Antibodies may be murine, human, humanized, chimeric, or derived from other species. An antibody is a protein generated by the immune system that is capable of recognizing and binding to a specific antigen (Janeway 2001). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1 IgG2, IgG3, IgG4, IgA1 and IgA2) or subclass of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term ‘monoclonal antibody’ as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler 1975, or may be made by recombinant DNA methods (see, US 4816567). The monoclonal antibodies may also be isolated from phage antibody libraries using the techniques described in Clackson 1991, Marks 1991 or from transgenic mice carrying a fully human immunoglobulin system (Lonberg 2008).

The monoclonal antibodies herein specifically include chimeric antibodies, humanized antibodies and human antibodies.

Examples of cell binding agents include those agents described for use in WO 2007/085930, which is incorporated herein.

Tumour-associate antigens and cognate antibodies for use in embodiments of the present invention are listed below, and are described in more detail on pages 14 to 86 of WO 2017/186894, which is incorporated herein.
(1) BMPR1B (bone morphogenetic protein receptor-type IB)
(2) E16 (LAT1, SLC7A5)
(3) STEAP1 (six transmembrane epithelial antigen of prostate)
(4) 0772P (CA125, MU016)
(5) MPF (MPF, MSLN, SMR, megakaryocyte potentiating factor, mesothelin)
(6) Napi3b (NAPI-3B, NPTIIb, SLC34A2, solute carrier family 34 (sodium phosphate), member 2, type II sodium-dependent phosphate transporter 3b)
(7) Sema 5b (FLJ10372, KIAA1445, Mm.42015, SEMA5B, SEMAG, Semaphorin 5b Hlog, 25 sema domain, seven thrombospondin repeats (type 1 and type 1-like), transmembrane domain (TM) and short cytoplasmic domain, (semaphorin) 5B)
(8) PSCA hlg (2700050C12Rik, C530008016Rik, RIKEN cDNA 2700050012, RIKEN cDNA 2700050012 gene)
(9) ETBR (Endothelin type B receptor)
(10) MSG783 (RNF124, hypothetical protein FLJ20315)
(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein)
(12) TrpM4 (BR22450, FLJ20041, TRPM4, TRPM4B, transient receptor potential cation 5 channel, subfamily M, member 4)
(13) CRIPTO (CR, CR1, CRGF, CRIPTO, TDGF1, teratocarcinoma-derived growth factor)
(14) CD21 (CR2 (Complement receptor 2) or C3DR (C3d/Epstein Barr virus receptor) or Hs.73792)
(15) CD79b (CD79B, CD7913, IGb (immunoglobulin-associated beta), B29)
(16) FcRH2 (IFGP4, IRTA4, SPAP1A (SH2 domain containing phosphatase anchor protein 1a), SPAP1B, SPAP1C)
(17) HER2 (ErbB2)
(18) NCA (CEACAM6)
(19) MDP (DPEP1)
(20) IL20R-alpha (IL20Ra, ZCYTOR7)
(21) Brevican (BCAN, BEHAB)
(22) EphB2R (DRT, ERK, Hek5, EPHT3, Tyro5)
(23) ASLG659 (B7h)
(24) PSCA (Prostate stem cell antigen precursor)
(25) GEDA
(26) BAFF-R (B cell -activating factor receptor, BLyS receptor 3, BR3)
(27) CD22 (B-cell receptor CD22-B isoform, BL-CAM, Lyb-8, Lyb8, SIGLEC-2, FLJ22814)
(27a) CD22 (CD22 molecule)
(28) CD79a (CD79A, CD79alpha), immunoglobulin-associated alpha, a B cell-specific protein that covalently interacts with Ig beta (CD79B) and forms a complex on the surface with Ig M molecules, transduces a signal involved in B-cell differentiation), pl: 4.84, MW: 25028 TM: 2 [P] Gene Chromosome: 19q13.2).
(29) CXCRS (Burkitt's lymphoma receptor 1, a G protein-coupled receptor that is activated by the CXCL13 chemokine, functions in lymphocyte migration and humoral defense, plays a 10 role in HIV-2 infection and perhaps development of AIDS, lymphoma; myeloma, and leukemia); 372 aa, pl: 8.54 MW: 41959 TM: 7 [P] Gene Chromosome: 11q23.3,
(30) HLA-DOB (Beta subunit of MHC class II molecule (la antigen) that binds peptides and 20 presents them to CD4+ T lymphocytes); 273 aa, pl: 6.56, MW: 30820.TM: 1 [p] Gene Chromosome: 6p21.3)
(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pl: 7.63, MW: 47206 TM: 1 [P] Gene Chromosome: 17p13.3).
(32) CD72 (B-cell differentiation antigen CD72, Lyb-2); 359 aa, pl: 8.66, MW: 40225, TM: 1 5 [P] Gene Chromosome: 9p13.3).
(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis); 661 aa, pl: 6.20, MW: 74147 TM: 1 [P] Gene Chromosome: 5q12).
(34) FcRH1 (Fc receptor-like protein 1, a putative receptor for the immunoglobulin Fc domain that contains C2 type Ig-like and ITAM domains, may have a role in B-lymphocyte 20 differentiation); 429 aa, pl: 5.28, MW: 46925 TM: 1 [P] Gene Chromosome: 1q21-1q22)
(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pl: 6.88, MW: 106468, TM: 1 [P] Gene Chromosome: 1q21)
(36) TENB2 (TMEFF2, tomoregulin, TPEF, HPP1, TR, putative transmembrane 35 proteoglycan, related to the EGF/heregulin family of growth factors and follistatin); 374 aa)
(37) PSMA-FOLH1 (Folate hydrolase (prostate-specific membrane antigen) 1)
(38) SST (Somatostatin Receptor; note that there are5 subtypes)
(38.1) SSTR2 (Somatostatin receptor 2)
(38.2) SSTR5 (Somatostatin receptor 5)
(38.3) SSTR1
(38,4) SSTR3
(38.5) SSTR4

### AvB6 — Both Subunits (39+40)

(39) ITGAV (Integrin, alpha V)
(40) ITGB6 (Integrin, beta 6)
(41) CEACAM5 (Carcinoembryonic antigen-related cell adhesion molecule 5)
(42) MET (met proto-oncogene; hepatocyte growth factor receptor)
(43) MUCI (Mucin 1, cell surface associated)
(44) CA9 (Carbonic anhydrase IX)
(45) EGFRvIII (Epidermal growth factor receptor (EGFR), transcript variant 3,
(46) CD33 (CD33 molecule)
(47) CD19 (CD19 molecule)
(48) IL2RA (Interleukin 2 receptor, alpha); NCBI Reference Sequence: NM_00417.2);
(49) AXL (AXL receptor tyrosine kinase)
(50) CD30-TNFRSF8 (Tumor necrosis factor receptor superfamily, member 8)
(51) BCMA (B-cell maturation antigen)-TNFRSFI7 (Tumor necrosis factor receptor superfamily, member 17)
(52) CT Ags-CTA (Cancer Testis Antigens)
(53) CD174 (Lewis Y) FUT3 (fucosyltransferase 3 (galactoside 3(4)-L-fucosyltransferase, Lewis blood group)
(54) CLEC14A (C-type lectin domain family 14, member A; Genbank accession no. NM175060)
(55) GRP78 HSPAS (heat shock 70 kDa protein 5 (glucose-regulated protein, 78 kDa)
(56) CD70 (CD70 molecule) LO8096
(57) Stem Cell specific antigens. For example:
   5T4 (see entry (63) below)
   CD25 (see entry (48) above)
   CD32
   LGRS/GPR49
   Prominin/CD133
(58) ASG-5
(59) ENPP3 (Ectonucleotide pyrophosphatase/phosphodiesterase 3)
(60) PRR4 (Praline rich 4 (lacrimal))
(61) GCC-GUCY2C (guanylate cyclase 2C (heat stable enterotoxin receptor)
(62) Liv-1-SLC39A6 (Solute carrier family 39 (zinc transporter), member 6)
(63) 5T4, Trophoblast glycoprotein, TPBG-TPBG (trophoblast glycoprotein)
(64) CD56 NCMA1 (Neural cell adhesion molecule 1)
(65) CanAg (Tumor associated antigen CA242)
(66) FOLR1 (Folate Receptor 1)
(67) GPNMB (Glycoprotein (transmembrane) nmb)
(68) TIM-1-HAVCR1 (Hepatitis A virus cellular receptor 1)
(69) RG-1/Prostate tumor target Mindin-Mindin/RG-1
(70) B7-H4-VTCN1 (V-set domain containing T cell activation inhibitor 1
(71) PTK7 (PTK7 protein tyrosine kinase 7)
(72) CD37 (CD37 molecule)
(73) CD138-SDC1 (syndecan 1)
(74) CD74 (CD74 molecule ; major histocompatibility complex, class II invariant chain)
(75) Claudins-CLs (Claudins)
(76) EGFR (Epidermal growth factor receptor)
(77) Her3 (ErbB3)-ERBB3 (v-erb-b2 erythroblastic leukemia viral oncogene hamolog 3 (avian))
(78) RON-MST1R (macrophage stimulating 1 receptor (c-met-related tyrosine kinase))
(79) EPHA2 (EPH receptor A2)
(80) CD20-MS4A1 (membrane-spanning 4-domains, subfamily A, member 1)
(81) Tenascin C-TNC (Tenascin C)
(82) FAP (Fibroblast activation protein, alpha)
(83) DKK-1 (Dickkopf 1 homolog (Xenopus laevis)
(84) CD52 (CD52 molecule)
(85) CS1-SLAM F7 (SLAM family member 7)
(86) Endoglin-ENG (Endoglin)
(87) Annexin A1-ANXA1 (Annexin A1)
(88) V-CAM (CD106) VCAM1 (Vascular cell adhesion molecule 1)

An additional tumour-associate antigen and cognate antibodies of interest are:
(89) ASCT2 (ASC transporter 2, also known as SLC1A5),

ASCT2 antibodies are described in WO 2018/089393, which is incorporated herein by reference

The cell binding agent may be labelled, for example to aid detection or purification of the agent either prior to incorporation as a conjugate, or as part of the conjugate. The label may be a biotin label. In another embodiment, the cell binding agent may be labelled with a radioisotope.

### Site-Specific Antibodies for Conjugation

The modification of antibodies to provide specific sites for conjugation is known. In some embodiments, the antibody or antibody fragment is a cysteine-engineered antibody. Of particular relevance to the present invention is the modification of antibodies to to have cysteine inserted between the 239 and 240 positions, as described in Dimasi 2017.

### Payloads

The payloads may be a drug molecule or a prodrug thereof. In some embodiments, the drug is selected from the group consisting of pharmaceutically active compounds, in particular low to medium molecular weight compounds (e.g. about 200 to about 2500 Da, preferably about 300 to about 1750 Da). In a further preferred embodiment, the drug is selected from the group consisting of cytotoxins, antiviral agents, antibacterials agents, peptides and oligonucleotides. Examples of cytotoxins include colchicine, vinca alkaloids, anthracyclines, camptothecins, doxorubicin, daunorubicin, taxanes, calicheamycins, tubulysins, irinotecans, an inhibitory peptide, amanitin, deBouganin, duocarmycins, maytansines, pyrrolobenzodiazepines (including dimers thereof) or auristatins. Preferred drugs include vinca alkaloids, anthracyclines, camptothecins, taxanes, tubulysins, amanitin, duocarmycins, maytansines, pyrrolobenzodiazepines (including dimers thereof) and auristatins.

Of particular interest are pyrrolobenzodiazepines (including dimers thereof) and auristatins.

Such cytotoxc drugs are extensively described, along with groups for linking them to cell binding agents, such as antibodies. Reference is made to Dosio 2011 and Beck 2017. Such drug-linkers form the basis of DL-1 and DL-2 used in the present invention, with modifications such that drug-linker intermediates used to make the drug-linkers of the fifth aspect of the invention terminate in —N₃ (i.e. DL-1-N₃) and amino-oxy, —O—NH₂ (i.e. DL-2-O—NH₂). These terminal groups allow for orthogonal reaction with the functional groups on the tri-functional linker moiety.

### Pyrrolobenzodiazepines

Dimer PBD (pyrrolobenzodiazepine) compounds having linker groups for connection to a cell binding agent, such as an antibody, are described in WO 2011/130598. The linker in these compounds is attached to one of the available N10 positions, and are generally cleaved by action of an enzyme on the linker group, Other disclosures of dimer PBD compounds linked by the N10 position include WO2013/055987, WO 2014/057074, WO2014/096368, WO2015/095124, WO 2015/052322, WO2016/044560, WO2017/137553, WO2017/137555, WO2018/069490, WO2018/192944, and WO2018/146188. WO2018/146188 discloses antibody drug conjugates comprising acyl sulfamides as part of the linker.

Disclosures of dimer PBD compounds linked by a substituent on the C2 position include WO2011/130613, WO2011/130616, WO2013/053873, WO2013/053871, WO2013/041606, WO2013/055993, WO2013/055990, WO2014/057073, WO2014/096365, WO2015/052321, WO2017/129652, WO2017/186894 and WO2018/091646.

Disclosures of dimer PBD compounds linked by a substituent on the C7 position include WO2014/140174 and WO2016/037644.

WO 2007/085930 describes the preparation of dimer PBD compounds having linker groups for connection to a cell binding agent, such as an antibody. The linker is present in the bridge linking the monomer PBD units of the dimer. Other disclosures of dimer PBD compounds have the linker in the bridge include WO2014/159981, WO2014/140862 and WO2016/038383.

In some embodiments of the present invention, DL-1-N₃ is SG3457:

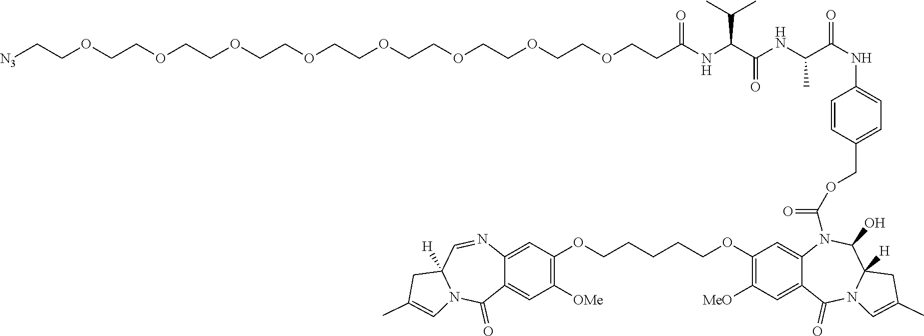 [SMILES: COc1cc2c(cc1OCCCCCOc1cc3c(cc1OC)C(=O)N1C=C(C)C[C@H]1[C@H](O)N3C(=O)OCc1ccc(NC(=O)[C@H](C)NC(=O)[C@@H](NC(=O)CCOCCOCCOCCOCCOCCOCCOCCOCCN=[N+]=[N-])C(C)C)cc1)N=C[C@@H]1CC(C)=CN1C2=O]

### Auristatins

WO2002/088172 describes the auristatins MMAE and MMAF. WO2004/010957 and WO2009/117531 and WO2014/093379, amongst others, describes auristatin drug linker conjugates.

In some embodiments of the present invention, DL-2-O—NH₂ is O-vc-PAB-MMAE:

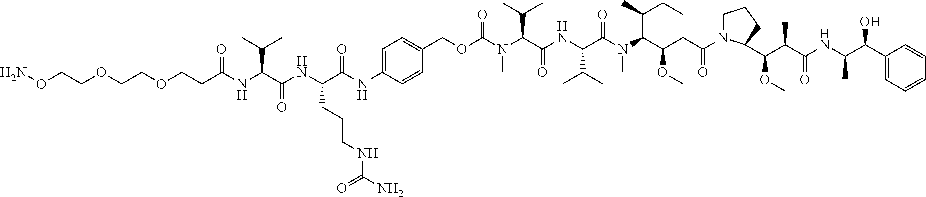 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCON)C(C)C)cc1)C(C)C]

This molecule is analogous to that described in Thompson 2015.

In some embodiments where two payloads/drugs are attached to the linker, preferred that these have different mechanisms of action.

### X

X is —(C(═O)—NH)ₓₐ—(CH₂)_{xb}—(CH₄O)_{xc}—(CH₂)_{xd}—^{≡}, where xa is 0 or 1, xb is 0-3, xc is 0 to 4 and xd is 0-3; and when xc is not 0, xd cannot be 0. The indication “^{≡}” shows where X binds the group —C≡CH, or the groups derived from this.

In some embodiments, xa is 0.

In other embodiments, xa is 1.

In some embodiments, xb is 0.

In other embodiments, xb is 1.

In other embodiments, xb is 2.

In other embodiments, xb is 3.

In some embodiments, xc is 0, 2 or 4.

In other embodiments, xc is 0.

In other embodiments, xc is 1.

In other embodiments, xc is 2.

In other embodiments, xc is 3.

In other embodiments, xc is 4.

In some embodiments, xd is 0.

In other embodiments, xd is 1.

In other embodiments, xd is 2.

In other embodiments, xd is 3.

In some embodiments, all of xa, xb, xc and xd are 0, i.e. X is a single bond.

In some embodiments, xa is 0, xb is 0-3, xc is 1 to 4 and xd is 1-3. In some of these embodiments, xa is 0, xb is 1, xc is 2 or 4 and xd is 2. In one of these embodiments, xa is 0, xb is 1, xc is 2 and xd is 2, i.e. X is:

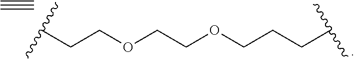

In some embodiments, xa is 1, xb is 0, xc is 0 and xd is 1-3. In one of these embodiments, xa is 1, xb is 0, xc is 0 and xd is 1, i.e. X is:

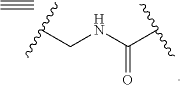

### Y

Y is —(C(═O)—NH)_{ya}—(CH₂)_{yb}—(C₂H₄O)_{yc}—(CH₂)_{yd}—(NH—C(═O))_{ye}—(CH₂)_{yf}—^{C═OMe}, where ya is 0 or 1, yb is 0-3, yc is 0 to 4, yd is 0-3, ye is 0 or 1, and yf is 0-3, and when yc and ye are both not 0, yd cannot be 0. The indication “^{C═OMe}” shows where Y binds the group —C═OMe, or the groups derived from this.

In some embodiments, ya is 0.

In other embodiments, ya is 1.

In some embodiments, yb is 0.

In other embodiments, yb is 1.

In other embodiments, yb is 2.

In other embodiments, yb is 3.

In some embodiments, yc is 0, 2 or 4

In other embodiments, yc is 0.

In other embodiments, yc is 1.

In other embodiments, yc is 2.

In other embodiments, yc is 3.

In other embodiments, yc is 4.

In some embodiments, yd is 0.

In other embodiments, yd is 1.

In other embodiments, yd is 2.

In other embodiments, yd is 3.

In some embodiments, ye is 0.

In other embodiments, ye is 1.

In some embodiments, yf is 0.

In other embodiments, yf is 1.

In other embodiments, yf is 2.

In other embodiments, yf is 3.

In some embodiments, one of ya and ye is 1, and the other is 0. In other embodiments, both of ya and ye are 1. In other embodiment, both of ya and ye are 0.

In some embodiments, all of ya, yb, yc, yd, ye and yf are 0, i.e. Y is a single bond.

In some embodiments, ya is 1, yb is 0, yc is 1 to 4, yd is 1-3, ye is 1 and yf is 1-3. In some of these embodiments, ya is 1, yb is 0, yc is 2 or 4, yd is 2, ye is 1 and yf is 3. In one of these embodiments, ya is 1, yb is 0, yc is 2, yd is 2, ye is 1 and yf is 3, i.e. Y is:

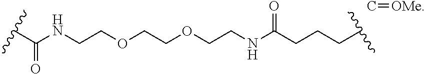

### X and Y

In some embodiments of the present invention X and Y are:

| | X | Y |
|---|---|---|
| (i) | Single bond | Single bond |
| (ii) | 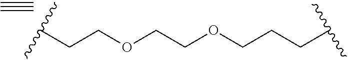 | Single bond |
| (iii) | 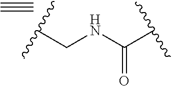 | 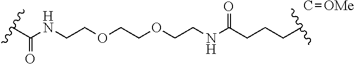 |

p

p represents the number of complete drug-linkers bound to each cell binding agent, e.g. antibody. If one payload is bound via the tri-functional moiety, then the p is the drug-loading. If two payloads are bound via the tri-functional moiety, then the drug-loading is 2p.

p may range from 1 to 10 per cell binding agent, i.e. where 1, 2, 3, 4, 5, 6, 7, 8, 9 and 10 moieties are covalently attached to the cell binding agent. Compositions of conjgates include collections of cell binding agents, e.g. antibodies, conjugated with a range of drugs, from 1 to 10.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblen 2004; Sanderson 2005). However, the distribution of p values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody-drug conjugates, p may be limited by the number of attachment sites on the antibody. For example, an antibody may have only one or several cysteine thiol groups, or may have only one or several sufficiently reactive thiol groups through which a linker may be attached. Higher drug loading may cause aggregation, insolubility, toxicity, or loss of cellular permeability of certain antibody-drug conjugates.

Typically, fewer than the theoretical maximum of drug linkers are conjugated to an antibody during a conjugation reaction. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (p) of an ADC may be controlled in several different manners, including: (i) limiting the molar excess of drug-linker relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody and which do not form intrachain or intermolecular disulfide linkages (Junutula 2008; Dornan 2009; U.S. Pat. Nos. 7,521,541; 7,723,485; WO2009/052249; Dimasi 2017). The engineered cysteine thiols may react with drug-linker reagents of the present invention which have thiol-reactive, electrophilic groups such as maleimide or alpha-halo amides to form ADC with cysteine engineered antibodies. The location of the drug moiety can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody. A drug loading near 2 can be achieved with near homogeneity of the conjugation product ADC.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the antibody-drug conjugate compositions of the invention include mixtures of antibody-drug conjugate compounds where the antibody has one or more drug moieties and where the drug moieties may be attached to the antibody at various amino acid residues.

In one embodiment, the average p is in the range 1 to 10. In some embodiments the range is selected from 2 to 10, 2 to 8, 2 to 6, and 4 to 10.

### Solvates

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

### Isomers

Certain compounds of the invention may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r- forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; a- and 8-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Enantiomerically enriched form” refers to a sample of a chiral substance whose enantiomeric ratio is greater than 50:50 but less than 100:0.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hvroxvazo, and nitro/aci-nitro.

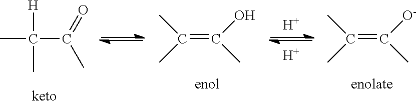

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Examples of isotopes that can be incorporated into compounds of the invention include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Various isotopically labeled compounds of the present invention, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic compounds of the invention may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the compounds of this invention any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

### Methods of Treatment

The conjugates of the present invention may be used in a method of therapy. Also provided is a method of treatment, comprising administering to a subject in need of treatment a therapeutically-effective amount of a conjugate of the third aspect of the invention (e.g of formula IIIa-1, IIIa-2, IIIb, IIIc-1, IIIc-2). The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A conjugate may be administered alone or in combination with other treatments, either simultaneously or sequentially dependent upon the condition to be treated. Examples of treatments and therapies include, but are not limited to, chemotherapy (the administration of active agents, including, e.g. drugs; surgery; and radiation therapy.

Pharmaceutical compositions according to the present invention, and for use in accordance with the present invention, may comprise, in addition to the active ingredient, i.e. a conjugate of the third aspect of the invention a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringers Injection, Lactated Ringers Injection. Preservatives, stabilisers, buffers, antioxidants and/or other additives may be included, as required.

The Conjugates can be used to treat proliferative disease and autoimmune disease. The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g., histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Other cancers of interest include, but are not limited to, haematological; malignancies such as leukemias and lymphomas, such as non-Hodgkin lymphoma, and subtypes such as DLBCL, marginal zone, mantle zone, and follicular, Hodgkin lymphoma, AML, and other cancers of B or T cell origin. Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Examples of autoimmune disease include the following: rheumatoid arthritis, autoimmune demyelinative diseases (e.g., multiple sclerosis, allergic encephalomyelitis), psoriatic arthritis, endocrine ophthalmopathy, uveoretinitis, systemic lupus erythematosus, myasthenia gravis, Graves' disease, glomerulonephritis, autoimmune hepatological disorder, inflammatory bowel disease (e.g., Crohn's disease), anaphylaxis, allergic reaction, Sjögren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), male and female autoimmune infertility, ankylosing spondolytis, ulcerative colitis, mixed connective tissue disease, polyarteritis nedosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the autoimmune disease is a disorder of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjögren's syndrome, Hashimoto's thyroiditis, Graves' disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes. In some embodiments, the autoimmunie disorder is a T cell-mediated immunological disorder.

### Methods of Treatment

The conjugates of the present invention may be used in a method of therapy. Also provided is a method of treatment, comprising administering to a subject in need of treatment a therapeutically-effective amount of a conjugate compound of the invention. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A compound of the invention may be administered alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor; Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), Ionafarnib (SARASAR''M, SCH 66336, Schering Plough), sorafenib (NEXAVARO, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil; chlornaphazine; chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1I, calicheamicin omegaI1 (Angew Chern. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis; dactinomycin, daunorubicin, detorubicin, 6-diazo-5-oxo-L-norleucine, morpholino-doxorubicin, cyanomorpholino-doxorubicin, 2-pyrroline-doxorubicin and deoxydoxorubicin), epirubicin, esorubicin, idarubicin, nemorubicin, marcellomycin, mitomycins such as mitomycin C, mycophenolic acid, nogalamycin ; olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); Ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMF©); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen); rituximab (RITUXAN®, Genentech/Biogen Idec), pertuzumab (OMNITARG™, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the conjugates of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motavizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, resyvizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Pharmaceutical compositions according to the present invention, and for use in accordance with the present invention, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection, Preservatives, stabilisers, buffers, antioxidants and/or other additives may be included, as required.

### Formulations

While it is possible for the conjugate compound to be used (e.g., administered) alone, it is often preferable to present it as a composition or formulation.

In one embodiment, the composition is a pharmaceutical composition (e.g., formulation, preparation, medicament) comprising a conjugate compound, as described herein, and a pharmaceutically acceptable carrier, diluent, or excipient.

In one embodiment, the composition is a pharmaceutical composition comprising at least one conjugate compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

Another aspect of the present invention pertains to methods of making a pharmaceutical composition comprising admixing at least one [¹¹C]-radiolabelled conjugate or conjugate-like compound, as defined herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like, Examples of suitable isotonic carriers for use in such formulations include Sodium Chloride Injection, Ringers Solution, or Lactated Ringer's Injection. Typically, the concentration of the active ingredient in the liquid is from about 1 ng/ml to about 10 μg/ml, for example from about 10 ng; ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the conjugate compound, and compositions comprising the conjugate compound, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

The dosage amounts described above may apply to the conjugate or to the effective amount of compound that is releasable after cleavage of the linker.

For the prevention or treatment of disease, the appropriate dosage of an ADC of the invention will depend on the type of disease to be treated, as defined above, the severity and course of the disease, whether the molecule is administered for preventive or therapeutic purposes, previous therapy, the patient's clinical history and response to the antibody, and the discretion of the attending physician. The molecule is suitably administered to the patient at one time or over a series of treatments. Depending on the type and severity of the disease, about 1 μg/kg to 100 mg/kg or more of molecule is an initial candidate dosage for administration to the patient, whether, for example, by one or more separate administrations, or by continuous infusion. For repeated administrations over several days or longer, depending on the condition, the treatment is sustained until a desired suppression of disease symptoms occurs. Other dosage regimens may be useful. The progress of this therapy is easily monitored by conventional techniques and assays.

### BRIEF DESCRIPTION OF THE FIGURES

FIG. 1 shows the effect on cell growth of conjugates of the invention, alongside controls;

FIG. 2 shows the effect on cell growth of conjugates of the invention on a different cell line, alongside controls.

### EXAMPLES

### General Information for Synthesis of the Compound A, B and C.

All reagents were purchased through VWR or Sigma Aldrich and were used without further purification. ¹H and ¹³C NMR spectra were obtained on a Bruker Ascend 400 spectrometer. Coupling constants are quoted in hertz (Hz). Mass Spectrometry was obtained using a Waters Acquity UPLC LCMS.

### General Information for the Synthesis of SG3457

Reaction progress was monitored by thin-layer chromatography (TLC) using Merck Kieselgel 60 F254 silica gel, with fluorescent indicator on aluminium plates. Visualisation of TLC was achieved with UV light. Flash column chromatography was performed using Merck Kieselgel 60 F254 silica gel. Extraction and chromatography solvents were bought and used without further purification from Fisher Scientific, U.K. All chemicals were purchased from Aldrich, Lancaster or BDH. Azido-dPEG®8-acid was purchased from Quanta biodesign. ¹H and ¹³C NMR spectra were obtained on a Bruker Avance 400 spectrometer. Coupling constants are quoted in hertz (Hz). Chemical shifts are recorded in parts per million (ppm) downfield from tetramethylsilane. Spin multiplicities are described as s (singlet), bs (broad singlet), d (doublet), t (triplet), q (quartet), p (pentuplet) and m (multiplet).

### LC/MS conditions

Method 1 (3 min run)

The HPLC (Shimadzu Nexera®/Prominence® LCMS-2020) was run using a mobile phase of water containing 0.1% formic acid (A) and acetonitrile containing 0.1% formic acid (B). Gradient: 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes.

Method 2 (15 min run)

The HPLC (Shimadzu Nexera®/Prominence® LCMS-2020) was run using a mobile phase of water containing 0.1% formic acid (A) and acetonitrile containing 0.1% formic acid (B). Gradient: 5% B held over 1.0 min, then increased from 5% B to 100% B over 9 min. The composition was held for 2 min at 100% B, then returned to 5% B in 10 seconds and held for 2 minutes 50 seconds. The total duration of the gradient run was 15.0 minutes.

Flow rate was 0.8 mL/minute (for 3-minute run) and 0.6 mL/minute (for 15-minute run). Detection was at 254 nm. Columns: Waters Acquity UPLC® BEH Shield RP18 1.7μm 2.1×50 mm at 50° C. fitted with Waters Acquity UPLC® BEH Shield RP18 VanGuard Pre-column, 130 A, 1.7 μm, 2.1 mm×5 mm (routine 3-minute run); and ACE Excel 2 C18-AR, 2 μ, 3.0×100 mm fitted with Waters Acquity UPLC® BEH Shield RP18 VanGuard Pre-column, 130 A, 1.7 μm, 2.1 mm×5 mm (15-minute run).

The preparative HPLC conditions were as follows: Reverse-phase ultra-fast high-performance liquid chromatography (UFLC) was carried out on a Shimazdzu Prominence® machine using a Phenomenex® Gemini NX 5 μ C18 column (at 50° C.) 150×21.2 mm. Eluents used were solvent A (H₂O with 0.1% formic acid) and solvent B (CH₃CN with 0.1% formic acid). All UFLC experiments were performed with gradient conditions: Initial composition 13% B increased to 100% B over a 15 minute period. The composition was held for 2 min at 100% B, then returned to 13% B in 0.1 min and held there for 2.9 min. The total duration of the gradient run was 20.0 minutes. Flow was 20.0 mL/minute and detection was at 254 and 280 nm.

### Example 1—Compound A (1)

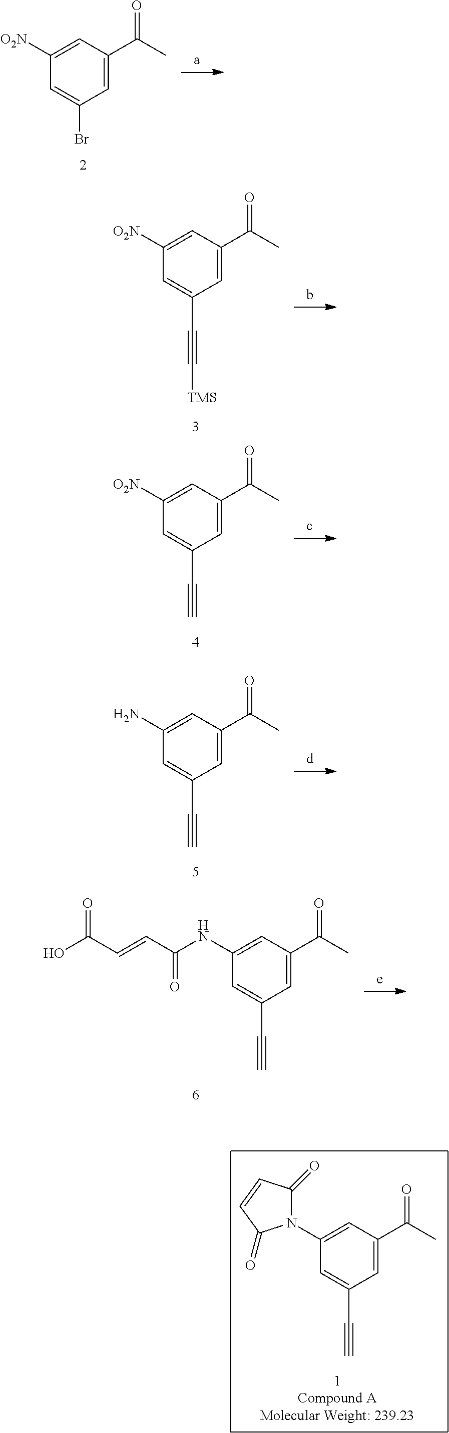 [SMILES: CC(=O)c1cc(Br)cc([N+](=O)[O-])c1 ; CC(=O)c1cc(C#C[Si](C)(C)C)cc([N+](=O)[O-])c1 ; C#Cc1cc(C(C)=O)cc([N+](=O)[O-])c1 ; C#Cc1cc(N)cc(C(C)=O)c1 ; C#Cc1cc(NC(=O)/C=C/C(=O)O)cc(C(C)=O)c1 ; C#Cc1cc(C(C)=O)cc(N2C(=O)C=CC2=O)c1]

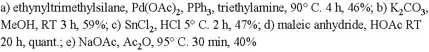

### 1-(3-nitro-5-((trimethylsily)ethynyl)phenyl) ethanone (3)

A turbid solution of 14 g 1-(3-bromo-5-nitrophenyl)ethanone 2 (57.4 mmol), 9.2 g ethynyltrimethylsilane (96.7 mmol), 0.75 g palladium(II) acetate (3.3 mmol), and 1.5 g triphenylphosphine (5.7 mmol) in 250 mL of trimethylamine was heated to gentle reflux under nitrogen for 4 h. The mixture was cooled, filtered, and concentrated under reduced pressure. To the residue was added 250 mL of saturated aqueous sodium bicarbonate. The mixture was extracted with DCM (3×150 mL). The organic solution was dried over sodium sulfate, concentrated to an oil, and purified by column chromatography (15:1 hexanes:ethyl acetate) to yield 3 as a yellowish solid (7 g, 46%). ¹H NMR (400 MHz, Chloroform-d) δ8.67 (dd, J=2.2, 1.6 Hz, 1H), 8.45 (dd, J=2.2, 1.5 Hz, 1H), 8.29 (t, J=1.5 Hz, 1H), 2.68 (s, 3H), 0.31 (s, OH), 0.28 (s, 9H). ¹³C NMR (101 MHz, CDCl₃) δ26.91, 77,37, 99.41, 101.33, 122.61, 125.95, 130.32, 136.93, 138.35, 148.50, 195,08.

### 1-(3-ethynyl-5-nitrophenyl) ethanone (4)

Compound 3 (7 g, 26.8 mmol) was treated with 0.5 g of anhydrous potassium carbonate (3.5 mmol) in 100 mL of methanol under nitrogen for 3 h at RT. The solvent was evaporated and the residue was mixed with 50 mL saturated aqueous sodium bicarbonate and extracted with DCM (3×150 mL). The solution was dried over sodium sulfate, concentrated to an oil, and purified by column chromatography (10:1 hexanes:ethyl acetate) to yield 4 as a slightly yellow solid (3 g, 59%). ¹H NMR (400 MHz, Chloroform-d) δ8.72 (dd, J=2.2, 1.6 Hz, 1H), 8.49 (dd, J=2.2, 1.5 Hz, 1H), 8.34 (t, J=1.5 Hz, 1H), 3.30 (s, 1H), 2.69 (s, 3H). ¹³C NMR (101 MHz, CDCl₃) δ26.89, 80.45, 81.30, 123.12, 124.91, 130.58, 137.14, 138.51, 194.91.

### 1-(3-amino-5-ethynylphenyl) ethanone (5)

To a cold solution (5° C.) of 4.5 g (20 mmol) stannous chloride in 6 mL concentrated hydrochloric acid was added compound 4. The reaction was stirred for 2 hours. The solution was cooled in an ice-salt bath and adjusted to pH 9 with saturated aqueous sodium carbonate. The solution was extracted with DOM (3×20 mL), concentrated, and purified by column chromatography (15:1 DCM:methanol) to yield 5 as a brown solid (0.4 g, 47%). ¹H NMR (400 MHz, Chloroform-d) δ7.44 (t, J=1.4 Hz, 1H), 7.24 (dd, J=2.4, 1.6 Hz, 1H), 6.97 (dd, J=2.4, 1.3 Hz, 1H), 3.84 (s, 2H), 3.07 (s, 1H), 2.55 (s, 3H). ¹³C NMR (101 MHz, CDCl₃) δ26.84, 77.59, 83.04, 114.65, 122.55, 122.91, 123.41, 138.41, 146.70, 197,65, HRMS (ESI) m/z calculated for C₁₀H₉NO [M+H]⁺ 160.08, found: 160,02.

### 1-(3-amino-5-ethynyl) phenyl maleimide (1)

A solution of 1-(3-amino-5-ethynylphenyl) ethanone 5 (500 mg, 3.14 mmol) and maleic anhydride (465 mg, 4.74 mmol) in acetic acid (20 mL) was stirred at room temperature for 20 h. The precipitated solid was collected by filtration, washed with hexane (3×20 mL), and dried in air to yield 6 as a beige solid, which was used without further purification. A suspension of 6 (880 mg, 3.42 mmol) and anhydrous sodium acetate (290 mg, 3.54 mmol) in acetic anhydride (20 mL) was stirred at 95° C. for 30 min. The reaction mixture was poured over ice water (40 mL) and the resulting precipitated solid was collected by filtration, washed with cold water (2×10 mL) and dried in air to provide pure 1 (325 mg, 43% over two steps) as an off-white solid. ¹H NMR (400 MHz, Chloroform-d) δ8.04 (t, J=1.5 Hz, 1H), 7.95 (t, J=1.8 Hz, 1H), 7.70 (dd, J=2.1, 1.5 Hz, 1H), 6.90 (s, 2H), 3.19 (s, 1H), 2.62 (s, 3H). ¹³C NMR (101 MHz, CDCl₃) δ26.82, 79.58, 81.72, 124.04, 125.94, 131.26, 132.13, 133.40, 134.56, 138.26, 168.94, 196.15. HRMS (ESI) m/z calculated for C¹⁴H⁹NO₃ [M+H]⁺ 240.07, found: 240.06.

### Example 2—Compound B (14)

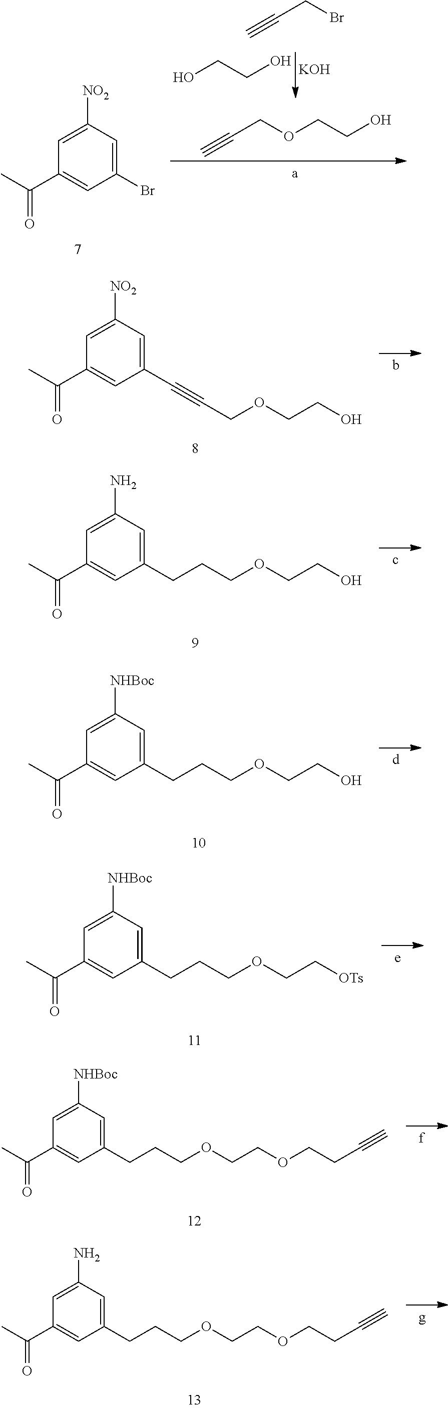

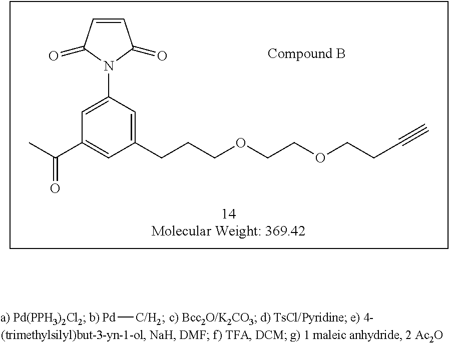

### 2-(Propargyloxy)ethanol

Propargyl bromide (80 wt % in toluene, 20 g of solution, 134.5 mmol, 1 equiv.) was added dropwise to a stirred solution of KOH (15.09 g, 269 mmol, 2 equiv.) in ethylene glycol (41.74 g, 672.5 mmol, 5 equiv.) and water (24 mL) at 4° C. The reaction mixture was allowed to warm to room temperature over 10-15 min and stirred further for 48 h. Water (15 mL) was added to the reaction mixture and extracted with ethyl acetate (4×100 mL). The combined organic extracts were dried with anhydrous Na₂SO₄ and concentrated under vacuum. The residue was purified by flash column chromatography over silica gel using 20-50% ethyl acetate in hexane as eluent. The fractions containing the product were combined and concentrated under vacuum to provide 2-(propargyloxy)ethanol (10.2g, 76%) as light yellow coloured viscous oil.

### 1-[3-[3-(2-hydroxyethoxy)prop-1-ynyl]-5-nitro-phenyl]ethanone (8)

A magnetically stirred solution of 3′-bromo-5′-nitroacetophenone (4.8 g, 19.67 mmol) and 2-(propargyloxy)ethanol (2.95 g, 29.51 mmol) in benzene (50 mL) and trimethylamine (30 mL) was degassed under vacuum and purged with nitrogen. This process was repeated 3 times and then Pd(PPh₃)₂Cl₂ was added. The mixture was again degassed twice under vacuum and purged with nitrogen. The reaction was heated to reflux for 2 h when TLC indicated reaction completion. The volatiles were removed under vacuum and the residue was purified by flash column chromatography over silica gel using 20-50% ethyl acetate in hexane as eluent, The fractions containing the product were combined and concentrated under vacuum to provide 8 as yellow coloured viscous oil (3.88 g, 75%).

### 1-[3-amino-5-[3-(2-hydroxyethxy)propyl]phenynl]ethanone (9)

To a solution of 8 (5.2 g, 19.75 mmol) in methanol (200 mL), Raney-Ni (1 g) was added and the mixture was hydrogenated in a Parr apparatus at 30 psi for 30-40 min. (The reaction progress was monitored by TLC and ¹H-NMR. Under these reaction conditions, the acetyl group is also reduced and it has been observed that longer reaction times leads exclusively to the over reduced compound. This hydrogenation is typically complete in about 30 min with some over reduced compound being formed as well). The reaction was filtered through a pad of celite and the pad was washed with methanol (2×50 mL). The filtrate and washings were combined and concentrated under vacuum. The crude residue was purified by flash column chromatography over silica gel using 20-50% ethyl acetate in hexane as eluent. The fractions containing the product were combined and concentrated under vacuum to provide 9 as a yellow coloured viscous oil (3.28 g, 70%).

### tert-butyl N-[3-acetyl-5-[3-(2-hydroxyethoxy)propyl]phenyl]carbamate (10)

A solution of 9 (3.6 g, 15.17 mmol) and potassium carbonate (8.4 g, 60.68 mmol, 4 equiv.) in THF/water (1:1, 50 mL) was stirred and cooled to 5-10° C. in an ice-water bath. Boc₂O (6.62 g, 30.34 mmol, 2 equiv.) was added dropwise over a period of 10 min. The reaction mixture was allowed to warm to room temperature over 10-15 min and stirred further for 20-22 h. THF was removed under vacuum. Water (20 mL) was added to the residue and extracted with ethyl acetate (4×50 mL). The combined organic extracts were dried with anhydrous Na₂SO₄ and concentrated under vacuum. The residue was purified by flash column chromatography over silica gel using 20-50% ethyl acetate in hexane as eluent. The fractions containing the product were combined and concentrated under vacuum to provide 10 as colorless viscous oil (4.25 g, 83%).

### tert-butyl N-[3-acetyl-5-[3-(2-hydroxyethoxy)propyl]phenyl]carbamate (11)

A stirred solution of 10 (3.7 g, 10.97 mmol) and pyridine (8.67 g, 109.7 mmol, 10 equiv.) in DCM (25 mL) was cooled to 0° C. in ice-salt bath and treated with p-toluenesulfonyl chloride (4.18 g, 21.94 mmol, 2 equiv.). The reaction mixture was allowed to warm to room temperature and stirred further for 20-22 h. The mixture was treated with saturated aq. NaHCO₃ solution (50 mL), stirred at room temperature for additional 20-30 min and extracted with DCM (3×100 mL). The combined organic layers were washed with 10% aq. citric acid (3×50 mL) and water (50 mL), dried (anhydrous Na₂SO₄), filtered and concentrated under vacuum. The crude residue was purified by flash column chromatography over silica gel using 25-50% ethyl acetate in hexane as eluent. The fractions containing the product were combined and concentrated under vacuum to provide 11 as colourless viscous oil (4.3 g, 80%).

### tert-butyl N-[3-acetyl-5-[3-(2-but-3-ynoxyethoxy)propyl]phenyl]carbamate (12)

A 100 mL 3-necked round-bottom flask, fitted with a water condenser, thermometer and addition funnel was charged with NaH (0.61 g, 15.27 mmol, 60% dispersion in mineral oil, 3 equiv), and purged with nitrogen. Anhydrous DMF (10 mL) was added via the addition funnel. The resulting slurry was stirred and cooled to 0° C. A solution of 4-trimethylsilyl-3-butyn-1-ol (2.17 g, 15.27 mmol, 3 equiv.) in anhydrous DMF (5 mL) was added dropwise at 0° C. After complete addition, the cooling bath was removed and the reaction mixture was stirred at room temperature for 1 h. A solution of 11 (2.5 g, 5.09 mmol) in anhydrous DMF (10 mL) was added dropwise at such a rate that the mixture temperature did not rise above 30° C. After complete addition, the reaction mixture was stirred further at room temperature for 1 h when TLC indicated reaction completion. The mixture was cooled to 0° C., quenched with saturated aq. NH₄Cl solution (100 mL) and extracted with ethyl acetate (2×150 mL). The combined organic layers were washed with water (2×50 mL), dried (anhydrous Na₂SO₄), filtered and concentrated under vacuum. The crude residue was purified by flash column chromatography over silica gel using 10-20% ethyl acetate in hexane as eluent. The fractions containing the product were combined and concentrated under vacuum to provide 12 as light yellow coloured viscous oil (1.4 g, 70%).

### 1-[3-amino-5-[3-(2-but-3-ynoxyethoxy)propyl]phenyl]ethanone (13)

A solution of 12 (0.55 g, 1.41 mmol) in DCM (5 mL) was stirred at room temperature. To this, was added trifluoroacetic acid (2.5 mL) in one portion. The mixture was stirred further at room temperature for 3-4 h, when TLC indicated reaction completion. The volatiles were removed under vacuum. The residue was treated with saturated aq. NaHCO₃ solution (20 mL) and extracted with DCM (3×40 mL). The combined organic layers were washed with water (2×20 mL), dried (anhydrous Na₂SO₄), filtered and concentrated under vacuum to provide 10 as dark yellow oil which was sufficiently pure and used as such for the next step (0.31 g, 76%).

### 1-[3-acetyl-5-[3-(2-but-3-ynoxyethoxy)propyl]phenyl]pyrrole-2,5-dione (14, Compound B)

A solution of 13 (0.31 g, 1.07 mmol) in acetic acid (6 mL) was stirred at room temperature. Maleic anhydride (0.21 g, 2.14 mmol, 2 equiv.) was added in one portion and the mixture stirred further at room temperature for 20-22 h. Excess acetic acid was removed under vacuum, the residue was treated with water (10 mL) and extracted with ethyl acetate (3×20 mL). The combined organic layers were washed with water (2×10 mL), dried (anhydrous Na₂SO₄), filtered and concentrated under vacuum. The crude residue was dissolved in acetic anhydride (6 mL) and stirred at room temperature. Anhydrous sodium acetate (0.044 g, 0.54 mmol) was added in one portion and the mixture was placed in an oil bath set at 80-85° C. The reaction mixture was stirred and heated for 30 min when TLC indicated reaction completion. The mixture was poured over ice-water (20 mL), stirred for 2-3 h and extracted with ethyl acetate (3×20 mL). The combined organic layers were washed with saturated aq. NaHCO₃ solution (3×10 mL) and water (10 mL), dried (anhydrous Na₂SO₄), filtered and concentrated under vacuum. The crude residue was purified by flash column chromatography over silica gel using 10-50% ethyl acetate in hexane as eluent. The fractions containing the product were combined and concentrated under vacuum to provide 14 as yellow coloured viscous oil (0.28 g, 71%).

### Compound C (19)

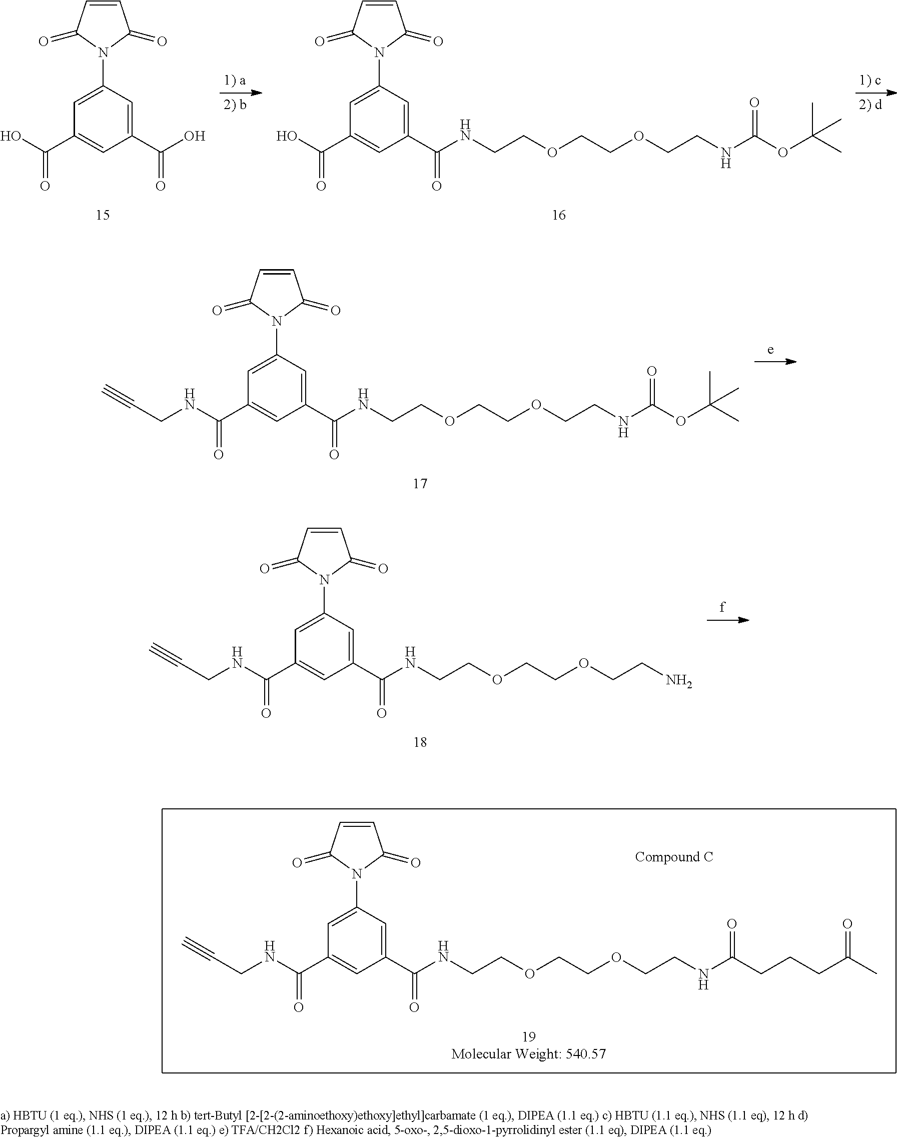

### 3-((2,2-dimethyl-4-oxo-3,8,11-trioxa-5-azatridecan-13-yl)carbarnoyl)-5-(2,5-dioxo-2,5-dihydro-1H-pyrrol-1-yl)benzoic acid (16)

A solution of compound 15 (5 g, 19.1 mmol) in DMF (25 mL) was treated with HBTU (7.3 g, 19.1 mmol) and NHS (2.2 g, 19.1 mmol). The solution was stirred overnight at room temperature followed by addition of tent-butyl [2-[2-(2-aminoethoxy)ethoxy]ethyl]carbamate (4.8 g, 19.1 mmol) and DIPEA (3.7 mL, 21.0 mmol). The resulting mixture was stirred at room temperature for 1 h followed by removal of solvent under reduced pressure. The resulting crude mixture was purified by reverse phase chromatography to yield 16 as a white solid (3.3 g, 36%). ¹H NMR (400 MHz, Methanol-d4) δ8.50 (s, 1H), 8.21 (s, 1H), 8.10 (s, 1H), 7.04 (s, 2H), 3.78-3.59 (m, 8H), 3.58-3.49 (m, 2H), 3.258-3.19 (m, 2H), 1.42 (s, 9H). ¹³C NMR (101 MHz, CDCl3) δ27.4, 39.7, 69.1, 69.7, 69.9, 78.7, 126.9, 128.9, 129.7, 134.3, 135.6, 166.6, 166.9, 169.4. MS (ESI) m/z calculated for C₂₃H₂₉N₃O₉ [M+H]⁺ 491.2, found: 492.2.

### tert-butyl (2-(2-(2-(3-(2,5-dioxo-2,5-dihydro-1H-pyrrol-1-yl)-5-(prop-2-yn-1-ylcarbamoyl)benzamido)ethoxy)ethoxy)ethyl)carbannate (17)

A solution of compound 16 (3 g, 6.1 mmol) in DMF (5 mL) was treated with HBTU (2.5 g, 6.7 mmol) and NHS (0.78 g, 6.7 mmol). The solution was stirred overnight at room temperature followed by addition of propargyl amine (0.47 mL, 7.3 mmol) and DIPEA (1.3 mL, 7.3 mmol). The resulting mixture was stirred at room temperature for 1 h followed by removal of solvent under reduced pressure. The resulting crude mixture was purified by reverse phase chromatography to yield 17 as a white solid (2.2 g, 69%). ¹H NMR (400 MHz, Methanol-d4) δ8.50 (s, 1H), 8.21 (s, 1H), 8.10 (s, 1H), 7.04 (s, 2H), 4.18 (s, 2H), 3.86-3.44 (m, 10H), 3.25-3.10 (m, 2H), 2.60 (s, 1H). ¹³C NMR (101 MHz, CDCl3) δ27.5, 28.9, 39.8, 69.1, 69.7, 71.3, 78.7, 79.4, 124.9, 127.7, 127.9, 132.4, 135.1, 135.7, 156.9, 166.3, 166.8, 169.5. MS (ESI) m/z calculated for C₂₆H₃₂N₄O₈ [M+H]⁺ 528.2, found: 529.2.

### N1-(2-(2-(2-aminoethoxy)ethoxy)ethyl)-5-(2,5-dioxo-2,5-dihydro-1H-pyrrol-1-yl)-N3-(prop-2-yn-1-yl)isophthalamide (18)

To a cooled solution of 17 (2.0 g, 3.8 mmol) in dichloromethane (2 mL) was added TFA (2 mL) in a dropwise manner. The resulting solution was stirred at room temperature for 2 h followed by removal of solvent under reduced pressure. The resulting crude mixture was purified by reverse phase chromatography to yield 18 as a white solid (1.5 g, 93%). ¹H NMR (400 MHz, Methanol-d4) δ8.32 (s, 1H), 8.00 (s, 2H), 6.98 (s, 2H), 4.02 (s, 2H), 3.78-3.59 (m, 8H), 3.58-3.49 (m, 2H), 2.54 (s, 2H), 1.28 (s, 9H). ¹³C NMR (101 MHz, CDCl3) δ30.6, 41.1, 68.1, 70.7, 71.5, 72.3, 80.9, 126.7, 129.3, 134.0, 136.0, 137.2, 168.0, 168.5, 171.2. MS (ESI) m/z calculated for C₂₁H₂₄N₄O₆ [M+H]⁺ 428.2, found: 429.2.

### 5-(2,5-dioxo-2,5-dihydro-1H-pyrrol-1-yl)-N1 -(2-(2-(2-(5-oxohexanarnido)ethoxy)ethoxy)ethyl)-N3-(prop-2-yn-1-yl)isophthalannide (19)

A solution of compound 18 (1.0 g, 2.3 mmol) in DMF (2 mL) was treated 2,5-dioxopyrrolidin-1-yl 5-oxohexanoate (0.63 g, 2.8 mmol) and DIPEA (0.4 mL, 2.8 mmol). The resulting mixture was stirred at room temperature for 1 h followed by removal of solvent under reduced pressure. The resulting crude mixture was purified by reverse phase chromatography to yield 2 as a white solid (0.58 g, 47%), MS (ESI) m/z calculated for C₂₇H₃₂N₄O₈ [M+H]⁺ 540.2, found: 541.2.

### Example 4—SG3457

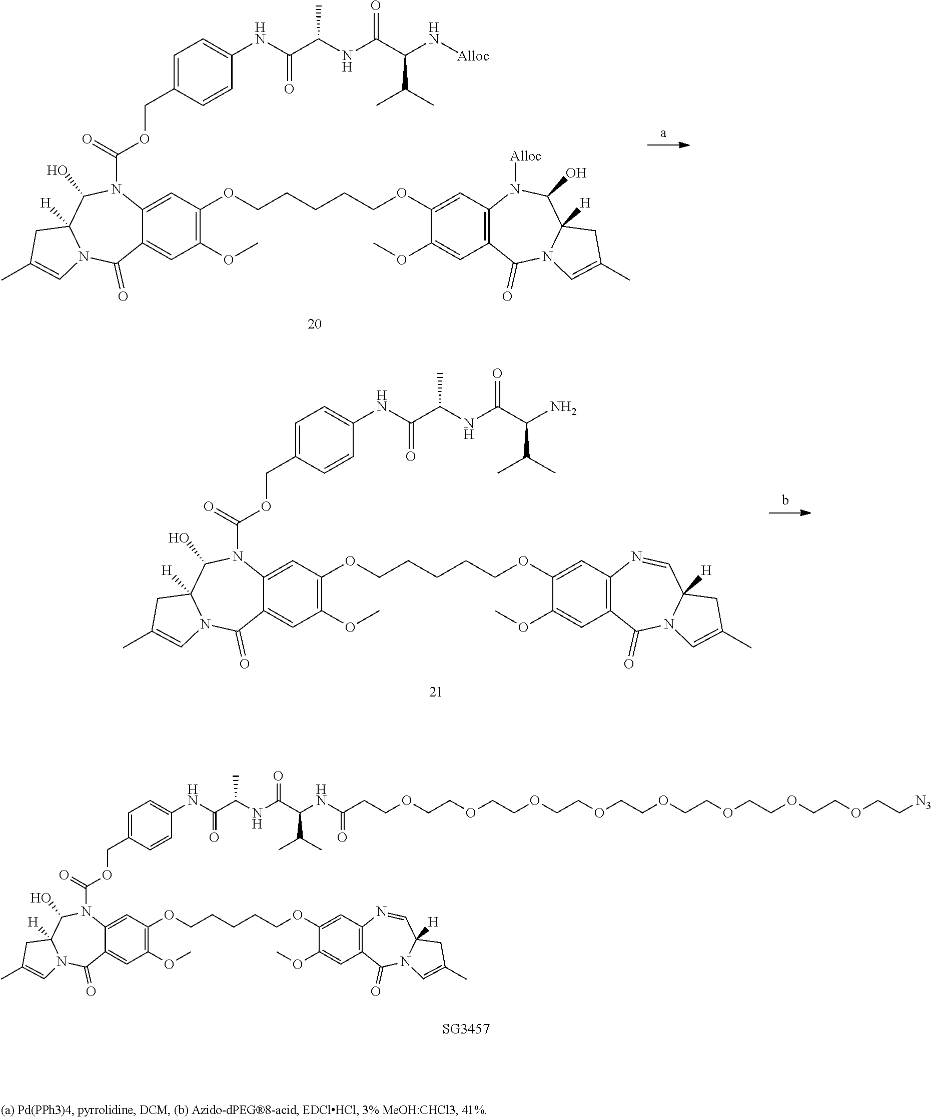

### (11S)-4-(2-(1-((1-amino-3-methyl-1-oxobutan-2-yl)amino)-1-oxopropan-2-yl)hydrazinyl)benzyl 11-hydroxy-7-methoxy-8-((5-(7-methoxy-2-methyl-5-oxo-5,11adihydro-1H-benzo[e]pyrrolo[1,2-a][1,4]diazepin-8-yl)oxy)pentyl)oxy)-2-methyl-5-oxo-11,11a-dihydro-1H-benzo[e]pyrrolo[1,2-a][1,4]diazepine-10(5H)-carboxylate (21)

20 (109 mg, 98 μmol, 1.0 eq.) and pyrrolidine (17.5 mg, 20.3 μl, 0.25 mmol, 2.5 eq.) in dry dichloromethane (5 mL) under an argon atmosphere. The reaction was stirred for 1 hour at room temperature diluted with dichloromethane and washed with saturated aqueous ammonium chloride solution (10 mL) and brine (10 mL). The organic phase was dried over magnesium sulphate, filtered and the dichloromethane removed by rotary evaporation under reduced pressure. The resulting product 21 was used without further purification. (see Tiberghien 2016)

### 4-((29S,32S)-1-azido-29-isopropyl-32-methyl-27,30-dioxo-3,6,9,12,15,18,21,24-octaoxa-28,31-diazatritriacontan-33-amido)benzyl (11S,11aS)-11-hydroxy-7-methoxy-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-11,11a-dihydro-1H-benzo[e]pyrrolo[1,2-a][1,4]diazepine-10(5H)-carboxylate (SG3457)

1-ethyl-3-(3′-dimethylaminopropyl)carbodiimide hydrochloride (EDCI.HCl, 18.9 mg, 98 μmol, 1.0 eq.) was added to a solution of compound 21 (91 mg, 98 μmol, 1.0 eq.) and Azido-dPEG®8-acid (46 mg, 98 μmol, 1.0 eq.) 3% methanol in chloroform (2 mL). The reaction mixture was stirred at room temperature for 18 h under an argon atmosphere. LC/MS and TLC analysis (5% MeOH in DCM) indicated the presence of a small amount of starting material. Additional portions of Azido-dPEG®8-acid (9.2 mg, 19.7 μmol, 0.2 eq.) and EDCI.HCl (3.8 mg, 19.7 μmol, 0.2 eq.) were added and the reaction continued for a further hour. The reaction mixture was diluted with dichloromethane (10 mL) and washed sequentially with water (10 mL) and brine (10 mL). The organic phase was dried over magnesium sulphate, filtered, and dichloromethane removed by rotary evaporation under reduced pressure. The crude material was purified by column chromatography (4 to 11% MeOH/CHCl₃) to give SG3457 as an off white foam (55 mg, 41%). LC/MS, RT=6.94 min (ES+) m/z (relative intensity) ([M+]+.1371, 25). [α]²²_{D}=+340.8° (c=1.0, CHCl₃). ¹H NMR (400 MHz, CDCl₃) δ8.82 (s, 1H), 8.87 (s, 1H), 7.68-7.58 (m, 2H), 7.5 (s, 1H), 7.35-7.4 (s, 1H), 7.43-7.1 (m, 4H), 6.82 (5, 1H), 6.74 (s, 1H), 6.68 (s, 1H), 6.48 (s, 1H), 5.77-5.73 (m, 1H), 5.35-5.25 and 4.8-4.7 (2×m 2H), 4.65-4.59 (m, 2H), 4.3-4.19 (m, 2H), 4.14-4.0 (m, 2H), 3.91 (s, 3H), 3.87 (s, 3H), 3.84-3.79 (m, 2H), 3.68-3.6 (m, 32H), 3.41-3.41 (m, 2H), 3.25-3.15 (m, 1H), 3.05-2.85 (m, 2H), 2.70-2.55 (m, 2H), 2.50-2.44 (m, 2H), 2.26-2.15 (m, 1H), 1.93-1.8 (m, 4H), 1.84 (s, 3H), 1.77 (s, 3H), 1.7-1.51 (m, 2H), 1.45-1.33 (m, 3H), 1.0-0.75 (m, 6H).

### General Procedure for Linker-Antibody Conjugation

Heterofunctional linker 19 was conjugated to the desired antibody in multiple steps. First, antibodies were mildly reduced to generate free thiols by adding 50 mM TCEP solution to 5 mL of 3.6 mg/mL antibody solution in 10 mM PBS, pH 7.4, 1 mM EDTA. The resulting solution was gently mixed at 37° C. for 1 h. Reduced antibody was transferred to a slide-a-lyzer dialysis cassette (10 K MWCO) and dialyzed against PBS, 1 mM EDTA, pH 7.4, 4° C. for 24 h with several buffer changes. Reduced antibody was oxidized to reform internal disulfides by addition of dehydroascorbic acid (50 mM stock in DMSO, 20 eq.) followed by gentle mixing for 4 h at room temperature. To the oxidized antibody solution was then added a solution of heterofunctional linker 1 (10 mM, DMSO, 4 eq.) The resulting reaction mixture was briefly vortexed and further incubated for the desired amount of time followed by addition of N-acetyl cysteine (10 μL of a 100 mM solution in water, 50 eq) and further incubation for 15 min to quench unreacted maleimide. All conjugation reactions were performed at room temperature (22° C.) under ambient atmosphere.

### General Procedure for Copper Catalyzed Click Reaction

Catalyst cocktail was prepared in a separate vial containing 0.64 mL of water and a solution of CuSO₄ (0.24 mL 100 mM) was added a solution of BTTAA ((4-{[bis-(1-tert-butyl-1H-[1,2,3]triazol-4-ylmethyl)-amino]-methyl}-[1,2,3]triazol-1-yl-acetic acid) (2.4 mL, 50 mM). To the resulting deep blue solution was added sodium ascorbate (0.72 mL, 500 mM) and the mixture vortexed until the color disappeared. The final concentration of this cocktail was as follows: CuSO4-6 mM; BTTAA—30 mM; sodium ascorbate—90 mM. In a separate tube containing a solution (10 mM PBS, pH 7.4) of antibody conjugated with linker 1 (8.5 mg/mL) was added the payload equipped with the azido group (10 mM, DMSO, 8 eq.). The final concentration of DMSO in the resultant solution was adjusted to 10% by adding free DMSO.

To this solution was added the catalyst cocktail to attain a final concentration of CuSO₄ as 1 mM. The resulting solution was gently mixed at room temperature for 5 h followed by purification using CHT (Ceramic Hydroxyapatite) column.

### General Procedure for Oxime Ligation

In a tube containing a solution (10 mM PBS, pH 7.2) of antibody conjugated with linker 1 (8.3 mg/mL) was added the payload equipped with the aminoxy group (10 mM, DMSO, 8 eq.). The final concentration of DMSO in the resultant solution was adjusted to 10% by adding free DMSO. To this solution was added the m-phenylenediamine catalyst (1 M, pH 7.2) to attain a final concentration of catalyst as 100 mM. The resulting solution was gently mixed at room temperature for 12 h followed by purification using CHT (Ceramic Hydroxyapatite) column.

### ADC Characterization-General

Reduced liquid chromatography mass spectrometry analysis (rLCMS), which was used to determine conjugation at the light or heavy chain and drug to antibody ratio (DAR), was performed on an Agilent 1290 series uHPLC coupled to an Agilent 6230 TOF. 2 μg of reduced antibodies or ADCs were loaded onto a Zorbax RRHD 300-Diphenyl (2.1×50 mm, 1.8 μm, Agilent) and eluted at a flow rate of 0.5 mL/min using a step gradient of 80% B after 2.1 min (mobile phase A: 0.1% Formic acid in water and mobile phase B: 0.1% Formic acid in acetonitrile). A positive time-of-flight MS scan was acquired and data collection and processing was carried out using MassHunter software (Agilent). Conjugation efficiencies were calculated based of Mass Spectrometry results.

In the case of both payloads being added, the oxime ligation was carried out prior to the CuAAC.

### Conjugates Synthesised

The antibodies used were trastuzumab engineered to carry a free cysteine inserted at position 239, and NIP228 carrying a cysteine insertion at position 239 (as an isotype control). See Dimasi, N., et al., Molecular Pharmaceutics, 2017, 14, 1501-1516 (DOI: 10.1021/acs. molpharmaceut.6b00995).

| | | | Unconjugated | Conjugated | Conjugation |
|---|---|---|---|---|---|
| Antibody | Linker (Mw) | Drug (Mw) | mass HC/LC | mass HC/LC | efficiency |
| Herceptin | Compound A | — | 50725.57/ | 50983.20/ | >95% |
| | (239.23) | | 23439.63 | 23439.63 | |
| NIP228 | Compound A | — | 50370.31/ | 50610.08/ | >95% |
| | (239.23) | | 23188.48 | 23188.44 | |
| Herceptin | Compound B | — | 50725.57/ | 51112.90/ | >95% |
| | (369.42) | | 23439.63 | 23439.62 | |
| NIP228 | Compound B | — | 50370.31/ | 50740.54/ | >95% |
| | (369.42) | | 23188.48 | 23188.65 | |
| Herceptin | Compound C | — | 50725.57/ | 51283.60/ | >95% |
| | (540.57) | | 23439.63 | 23439.40 | |
| NIP228 | Compound C | — | 50370.31/ | 50911.85/ | >95% |
| | (540.57) | | 23188.48 | 23188.46 | |
| Herceptin | Compound C | Aminooxy-Val-Cit- | 50725.57/ | 52565.65/ | >90% |
| | (540.57) | PABA-MMAE | 23439.63 | 23439.79 | |
| | | (1298.63) | | | |
| Herceptin | Compound C | SG3457 (1371.55) | 50725.57/ | 52828.71/ | >90% |
| | (540.57) | | 23439.63 | 23439.13 | |
| Herceptin | Compound C | SG3457 (1371.55) + | 50725.57/ | 53936.86/ | >90% |
| | (540.57) | Aminooxy-Val-Cit- | 23439.63 | 23439.68 | |
| | | PABA-MMAE | | | |
| | | (1298.63) | | | |
| NIP228 | Compound C | Aminooxy-Val-Cit- | 50370.31/ | 52209.38/ | >90% |
| | (540.57) | PABA-MMAE | 23188.48 | 23188.47 | |
| | | (1298.63) | | | |
| NIP228 | Compound C | SG3457 (1371.55) | 50370.31/ | 52471.41/ | >90% |
| | (540.57) | | 23188.48 | 23187.93 | |
| NIP228 | Compound C | SG3457 (1371.55) + | 50370.31/ | 53580.70/ | >90% |
| | (540.57) | Aminooxy-Val-Cit- | 23188.48 | 23188.14 | |
| | | PABA-MMAE | | | |
| | | (1298.63) | | | |

### Determination of In Vitro Cell Viability

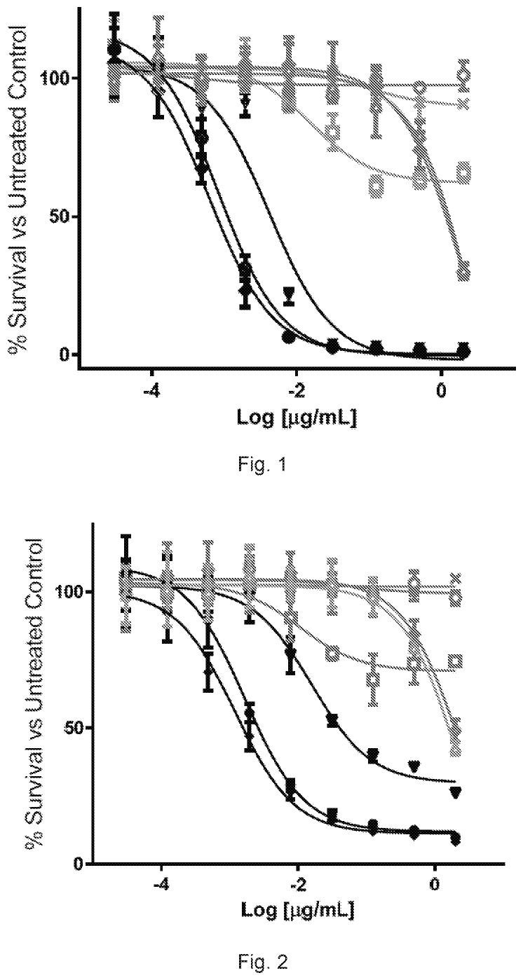

Human cancer cell lines SK-BR-3, BT-474, and MDA-MB-453 were seeded into white polystyrene tissue-culture treated 96-well plates (Costar) at a density of 3000 cells/well in RPMI +10% FBS (Invitrogen). On the following day, antibodies and ADCs were spiked into triplicate wells using an 8-point dose curve of 1:4 serial dilutions starting from 0.5 μg/mL. Cell viability was determined 6 days later using the Cell Titer-Glo Luminescent Cell Viability Assay kit (Promega) following the manufacturers protocol. Luminescence was measured using an EnVision 2104 Multilabel Reader (Perkin Elmer). Cell viability was calculated as a percentage of control untreated cells. Results of the in vitro cell viability assay are shown in FIG. 1 (SKBR3 (HER2+) 3000 cells/well) and FIG. 2 (MDA-MB-453 (HER2+) 3000 cells/wells), in which the symbols represent the following antibodies/conjugates:

| | |
|---|---|
| ∘ | NIP228-Unconjugated |
| □ | Herceptin-Unconjugated |
| x | NIP228-19-Aoa-vc-PAB-MMAE |
| ▾ | Herceptin-19-Aoa-vc-PAB-MMAE |
| Δ | NIP228-19-SG3457 |
| ♦ | Herceptin-19-SG3457 |
| ⋄ | NIP228-19-Aoa-vc-PAB-MMAE-SG3457 |
| ● | Herceptin-19-Aoa-vc-PAB-MMAE- |
| | SG3457 |

Unmodified antibodies and NIP228 ADCs were non-toxic to the MDA-MB-453 cells at the investigated concentration. Herceptin-19-SG3457 was found to be significantly more toxic than Herceptin-19-O-vc-PAB-M MAE. Dual drug conjugate (Herceptin-19-O-vc-PAB-MMAE-SG3457) was found to be equipotent to Herceptin-19-SG3457.

### REFERENCES

| | |
|---|---|
| Agarwal | Agarwal, P. and C. R. Bertozzi, Site-specific antibody-drug conjugates: the |
| 2015 | nexus of bioorthogonal chemistry, protein engineering, and drug development. |
| | Bioconjug Chem, 2015. 26 (2): p. 176-92. |
| Beck 2014 | Beck, A. and J. M. Reichert, Antibody-drug conjugates: present and future. |
| | MAbs, 2014. 6 (1): p. 15-7 |
| Beck 2017 | Beck, A, et al., Nat Rev Drug Discov. 2017 May; 16 (5): 315-337 (doi: |
| | 10.1038/nrd.2016.268) |
| Behrens | Behrens, C. R. and B. Liu, Methods for site-specific drug conjugation to antibodies. |
| 2014 | MAbs, 2014. 6 (1): p. 46-53. |
| Clackson | Clackson et al (1991) Nature, 352: 624-628 |
| 1991 | |
| Dimasi | Dimasi, N., et al., Molecular Pharmaceutics, 2017, 14, 1501-1516 (DOI: |
| 2017 | 10.1021/acs.molpharmaceut.6b00995). |
| Dornan | Dornan et al (2009) Blood 114 (13): 2721-2729 |
| 2009 | |
| Dosio | Dosio, F, et al., Toxins (Basel). 2011 July; 3 (7): 848-883 (doi: |
| 2011 | 10.3390/toxins3070848) |
| Fanale | Fanale, M. A., et al., Brentuximab Vedotin in the Front-Line Treatment of |
| 2014 | Patients With CD30(+)Peripheral T-Cell Lymphomas: Results of a Phase I |
| | Study. Journal of Clinical Oncology, 2014. 32 (28): p. 3137-3143 |
| Hamblett | Hamblett et al (2004) Clin. Cancer Res. 10: 7063-7070 |
| 2004 | |
| Janeway | Janeway, C., Travers, P., Walport, M., Shlomchik (2001) Immuno Biology, 5th Ed., |
| 2001 | Garland Publishing, New York |
| Junutula | Junutula, J. R., et al., Site-specific conjugation of a cytotoxic drug to an antibody |
| 2008 | improves the therapeutic index. Nat Biotechnol, 2008. 26 (8): p. 925-32. |
| Junutula | Junutula, et al., 2008 Nature Biotech., 26 (8): 925-932 |
| 2008 | |
| Kohler | Kohler et al (1975) Nature 256: 495 |
| 1975 | |
| Levengood | Levengood, M. R., et al., Orthogonal Cysteine Protection Enables |
| 2017 | Homogeneous Multi-Drug Antibody-Drug Conjugates. Angewandte Chemie |
| | (International Ed. in English), 2017. 56 (3): p. 733-737. |
| Li 2015 | Li, X., et al., Site-Specific Dual Antibody Conjugation via Engineered Cysteine and |
| | Selenocysteine Residues. Bioconjugate Chemistry, 2015. 26 (11): p. 2243-2248 |
| Loganzo | Loganzo, F., et al., Tumor Cells Chronically Treated with a Trastuzumab- |
| 2015 | Maytansinoid Antibody-Drug Conjugate Develop Varied Resistance Mechanisms but |
| | Respond to Alternate Treatments. Molecular Cancer Therapeutics, 2015. 14 (4): p. 952 |
| Lonberg | Lonberg (2008) Curr. Opinion 20 (4): 450-459 |
| 2008 | |
| Marks 1991 | Marks et al (1991) J. Mol. Biol., 222: 581-597 |
| Miller 2003 | Miller et al (2003) Jour. of Immunology 170: 4854-4861 |
| Panowski | Panowski, S., et al., Site-specific antibody drug conjugates for cancer therapy. |
| 2014 | MAbs, 2014. 6 (1): p. 34-45. |
| Sanderson | Sanderson et al (2005) Clin. Cancer Res. 11: 843-852 |
| 2005 | |
| Stern 2015 | Stern, H. M., et al., PTEN loss is associated with worse outcome in HER2- |
| | amplified breast cancer patients but is not associated with trastuzumab |
| | resistance. Clinical cancer research: an official journal of the American |
| | Association for Cancer Research, 2015. 21 (9): p. 2065-2074 |
| Tang 2016 | Tang, F., et al., One-pot N-glycosylation remodeling of IgG with non-natural |
| | sialylglycopeptides enables glycosite-specific and dual-payload antibody-drug |
| | conjugates. Organic & Biomolecular Chemistry, 2016. 14 (40): p. 9501-9518 |
| Tang 2017 | Tang, Y., et al., Real-Time Analysis on Drug-Antibody Ratio of Antibody-Drug |
| | Conjugates for Synthesis, Process Optimization, and Quality Control. |
| | Scientific Reports, 2017. 7 (1): p. 7763 |
| Thompson | Thompson, P, et a., Bioconjugate Chem. 2015, 26, 10, 2085-2096 doi: |
| 2015 | 10.1021/acs.bioconjchem.5b00355 |
| Tiberghien | Tiberghien, A., et al., ACS Med Chem Lett. 2016 May 24; 7 (11): 983-987 |
| 2016 | |
| Younes | Younes, A., et al., Brentuximab vedotin combined with ABVD or AVD for |
| 2013 | patients with newly diagnosed Hodgkin's lymphoma: a phase 1, open-label, |
| | dose-escalation study. The Lancet Oncology. 2013 December, 14 (13): p. 1348-1356 |

### Embodiments of Invention

1. A compound of formula I:

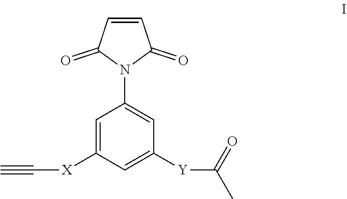

where:
X is —(C(═O)—NH)ₓₐ—(CH₂)_{xb}—(C₂H₄O)_{xc}—(CH₂)_{xd}—^{≡}, where xa is 0 or 1, xb is 0-3, xc is 0 to 4 and xd is 0-3; and
Y is —(C(═O)—NH)_{ya}—(CH₂)_{yb}—(C₂H₄O)_{yc}—(CH₂)_{yd}—(NH—C(═O))_{ye}—(CH₂)_{yf}—^{C═OMe}, where ya is 0 or 1, yb is 0-3, yc is 0 to 4, yd is 0-3, ye is 0 or 1, and yf is 0-3,
with the provisos that:
(i) when xc is not 0, xd cannot be 0; and
(ii) when yc and ye are both not 0, yd cannot be 0.

2. A compound according to embodiment 1, wherein xa is 0.

3. A compound according to embodiment 1, wherein xa is 1.

4. A compound according to any one of embodiments 1 to 3, wherein xb is 0.

5. A compound according to any one of embodiments 1 to 3, wherein xb is 1.

6. A compound according to any one of embodiments 1 to 3, wherein xb is 2.

7. A compound according to any one of embodiments 1 to 3, wherein xb is 3.

8. A compound according to any one of embodiments 1 to 7, wherein xc is 0, 2 or 4.

9. A compound according to any one of embodiments 1 to 7, wherein xc is 0.

10. A compound according to any one of embodiments 1 to 7, wherein xc is 1.

11. A compound according to any one of embodiments 1 to 7, wherein xc is 2.

12. A compound according to any one of embodiments 1 to 7, wherein xc is 3.

13. A compound according to any one of embodiments 1 to 7, wherein xc is 4.

14. A compound according to any one of embodiments 1 to 13, wherein xd is 0.

15. A compound according to any one of embodiments 1 to 13, wherein xd is 1.

16. A compound according to any one of embodiments 1 to 13, wherein xd is 2.

17. A compound according to any one of embodiments 1 to 13, wherein xd is 3.

18. A compound according to embodiment 1, wherein all of xa, xb, xc and xd are 0.

19. A compound according to embodiment 1, wherein xa is 0, xb is 0-3, xc is 1 to 4 and xd is 1-3.

20. A compound according to embodiment 19, wherein xa is 0, xb is 1, xc is 2 or 4 and xd is 2.

21. A compound according to embodiment 20, wherein xa is is 0, xb is 1, xc is 2 and xd is 2.

22. A compound according to embodiment 1, wherein xa is 1, xb is 0, xc is 0 and xd is 1-3.

23. A compound according to embodiment 1, wherein xa is 1, xb is 0, xc is 0 and xd is 1.

24. A compound according to any one of embodiments 1 to 23, wherein ya is 0.

25. A compound according to any one of embodiments 1 to 23, wherein ya is 1.

26. A compound according to any one of embodiments 1 to 25, wherein yb is 0.

27. A compound according to any one of embodiments 1 to 25, wherein yb is 1.

28. A compound according to any one of embodiments 1 to 25, wherein yb is 2.

29. A compound according to any one of embodiments 1 to 25, wherein yb is 3.

30. A compound according to any one of embodiments 1 to 29, wherein yc is 0, 2 or 4

31. A compound according to any one of embodiments 1 to 29, wherein yc is 0.

32. A compound according to any one of embodiments 1 to 29, wherein yc is 1.

33. A compound according to any one of embodiments 1 to 29, wherein yc is 2.

34. A compound according to any one of embodiments 1 to 29, wherein yc is 3.

35. A compound according to any one of embodiments 1 to 29, wherein yc is 4.

36. A compound according to any one of embodiments 1 to 35, wherein yd is 0.

37. A compound according to any one of embodiments 1 to 35, wherein yd is 1.

38. A compound according to any one of embodiments 1 to 35, wherein yd is 2.

39. A compound according to any one of embodiments 1 to 35, wherein yd is 3.

40. A compound according to any one of embodiments 1 to 39, wherein ye is 0.

41. A compound according to any one of embodiments 1 to 39, wherein ye is 1.

42. A compound according to any one of embodiments 1 to 41, wherein yf is 0.

43. A compound according to any one of embodiments 1 to 41, wherein yf is 1.

44. A compound according to any one of embodiments 1 to 41, wherein yf is 2.

45. A compound according to any one of embodiments 1 to 41, wherein yf is 3.

46. A compound according to any one of embodiments 1 to 23, wherein one of ya and ye is 1, and the other is 0.

47. A compound according to any one of embodiments 1 to 23, wherein both of ya and ye are 1.

48. A compound according to any one of embodiments 1 to 23, wherein both of ya and ye are 0.

49. A compound according to any one of embodiments 1 to 23, wherein all of ya, yb, yc, yd, ye and yf are 0.

50. A compound according to any one of embodiments 1 to 23, wherein ya is 1, yb is 0, yc is 1 to 4, yd is 1-3, ye is 1 and yf is 1-3.

51. A compound according to embodiment 50, wherein ya is 1, yb is 0, yc is 2 or 4, yd is 2, ye is 1 and yf is 3.

52. A compound according to embodiment 50, wherein ya is 1, yb is 0, yc is 2, yd is 2, ye is 1 and yf is 3.

53. A compound according to embodiment 1, wherein X and Y are selected from:

| | X | Y |
|---|---|---|
| (i) | Single bond | Single bond |
| (ii) | 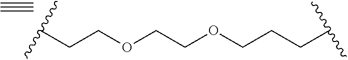 | Single bond |
| (iii) | 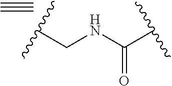 | 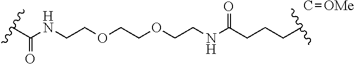 |

54. A linker between one or two payloads and a cell binding agent comprising one of the following moieties (IIa-1, IIa-2, IIb, IIc-1, IIc-2):

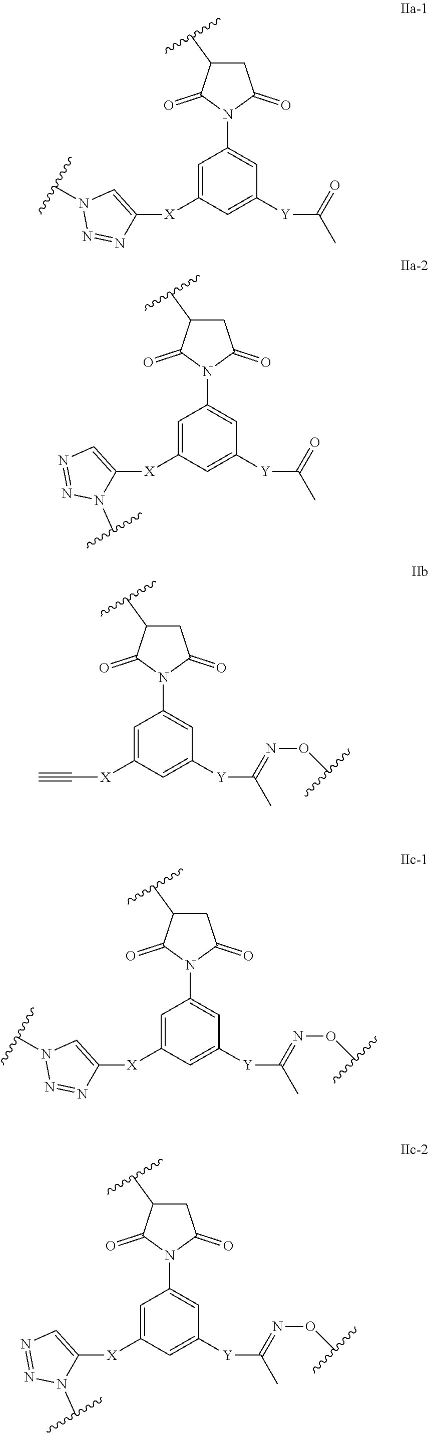

where X and Y are as defined in any one of embodiments 1 to 53

55. A conjugate of one of the following formulae (IIIa-1, III-2, IIIb, IIIc-1, IIIc-2):

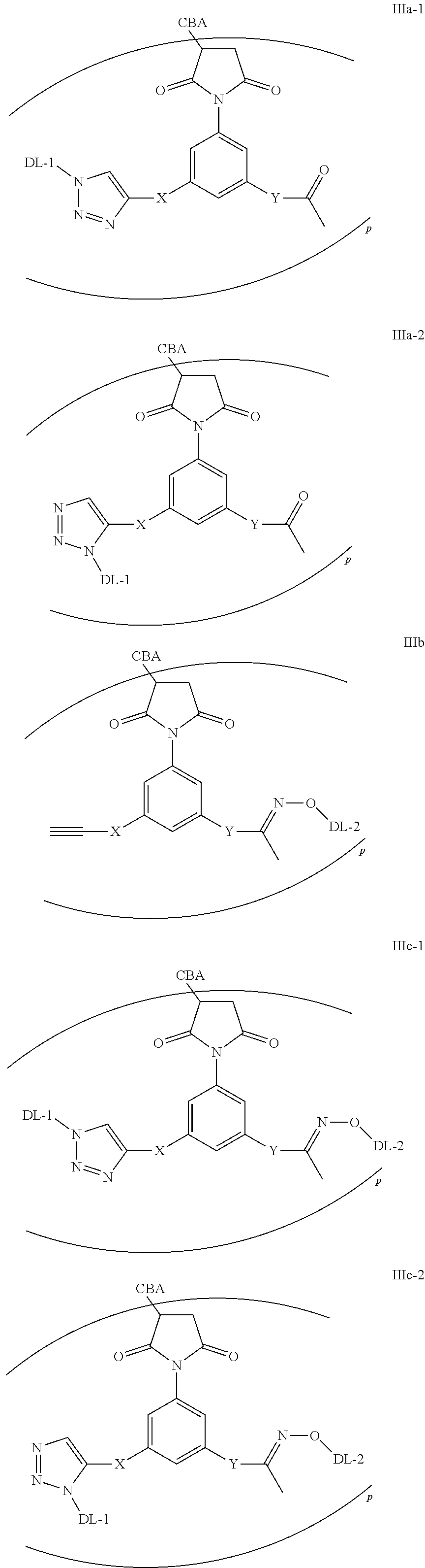

where X and Y are as defined in any one of embodiments 1 to 53, CBA is a cell binding agent, DL-1 is a first drug-linker moiety, DL-2 is a second drug-linker moiety, and p is from 1 to 10.

56. The conjugate according to embodiment 55, wherein the cell binding agent is an antibody or an active fragment thereof.

57. The conjugate according to embodiment 56, wherein the antibody or antibody fragment is an antibody or antibody fragment for a tumour-associated antigen.

58. The conjugate according to embodiment 57, wherein the antibody or antibody fragment is an antibody which binds to one or more tumor-associated antigens or cell-surface receptors selected from (1)-(89):
(1) BMPR1B;
(2) E16;
(3) STEAP1;
(4) 0772P;
(5) MPF;
(6) Napi3b;
(7) Sema 5b;
(8) PSCA hlg;
(9) ETBR;
(10) MSG783;
(11) STEAP2;
(12) TrpM4;
(13) CRIPTO;
(14) CD21;
(15) CD79b;
(16) FcRH2;
(17) HER2;
(18) NCA;
(19) MDP;
(20) IL20R-alpha;
(21) Brevican;
(22) EphB2R;
(23) ASLG659;
(24) PSCA;
(25) GEDA;
(26) BAFF-R;
(27) CD22;
(28) CD79a;
(29) CXCR5;
(30) HLA-DOB;
(31) P2X5;
(32) CD72;
(33) LY64;
(34) FcRH1;
(35) IRTA2;
(36) TENB2;
(37) PSMA-FOLH1;
(38) SST;
(38.1) SSTR2;
(38.2) SSTR5;
(38.3) SSTR1;
(38.4)SSTR3;
(38.5) SSTR4;
(39) ITGAV;
(40) ITGB6;
(41) CEACAM5;
(42) MET;
(43) MUC1;
(44) CA9;
(45) EGFRvill;
(46) CD33;
(47) CD19;
(48) IL2RA:
(49) AXL;
(50) CD30-TNFRSF8;
(51) BCMA TNFRSF17;
(52) CT Ags CTA;
(53) CD174 (Lewis Y)-FUT3;
(54) CLEC14A;
(55) GRP78 HSPAS;
(56) CD70;
(57) Stem Cell specific antigens;
(58) ASG-5;
(59) ENPP3;
(60) PRR4;
(61) GCC-GUCY2C;
(62) Liv-1-SLC39A6;
(63) 5T4;
(64) CD56 NCMA1;
(65) CanAg;
(66) FOLR1;
(67) GPNMB;
(68) TIM-1-HAVCR1;
(69) RG-1/Prostate tumor target Mindin Mindin/RG-1;
(70) B7-H4-VTCN1;
(71) PTK7;
(72) CD37;
(73) CD138-SDC1;
(74) CD74;
(75) Claudins-CLs;
(76) EGFR;
(77) Her3;
(78) RON-MST1R;
(79) EPHA2;
(80) CD20-MS4A1;
(81) Tenascin C-TNC;
(82) FAP;
(83) DKK-1;
(84) CD52;
(85) CS1-SLAM F7;
(86) Endoglin — ENG;
(87) Annexin A1-ANXA1;
(88) V-CAM (CD106)-VCAM1;
(89) ASCT2 (SLC1A5).

59. The conjugate according to any one of embodiments 56 to 58, wherein the antibody or antibody fragment is a cysteine-engineered antibody.

60. The conjugate according to embodiment 59, wherein the cysteine is inserted between the 239 and 240 positions.

61. The conjugate according to any one of embodiments 56 to 60, wherein the antibody or antibody fragment is to the tumor-associated antigen HER2.

62. The conjugate according to any one of embodiments 56 to 61, wherein p is an integer from 1 to 8.

63. The conjugate according to embodiment 62, wherein p is 2.

64. The conjugate according to embodiments 56 to 63, wherein drugs in the first drug-linker moiety and second drug-linker moiety (if present) are selected from pharmaceutically active compounds, which have a molecular weight between about 200 to about 2500 Da.

65. The conjugate according to embodiment 64, wherein the first drug-linker moiety and second drug-linker moiety (if present) are selected from pharmaceutically active compounds, which have a molecular weight between about 300 to about 1750 Da.

66. The conjugate according to embodiments 56 to 63, wherein drugs in the first drug-linker moiety and second drug-linker moiety (if present) are selected from the group consisting of cytotoxins, antiviral agents, antibacterials agents, peptides and oligonucleotides.

67. The conjugate according to embodiment 66, wherein a cytoxin is selected from the group consisting of colchicine, vinca alkaloids, anthracyclines, camptothecins, doxorubicin, daunorubicin, taxanes, calicheamycins, tubulysins, irinotecans, an inhibitory peptide, amanitin, deBouganin, duocarmycins, maytansines, pyrrolobenzodiazepines (including dimers thereof) and auristatins.

68. The conjugate according to embodiment 67, wherein a cytoxin is selected from the group consisting of vinca alkaloids, anthracyclines, camptothecins, taxanes, tubulysins, amanitin, duocarmycins, maytansines, pyrrolobenzodiazepines (including dimers thereof) and auristatins.

69. The conjugate according to embodiment 68, wherein a cytoxin is selected from the group consisting of pyrrolobenzodiazepines (including dimers thereof) and auristatins.

70. The conjugate according to embodiment 69, wherein DL-1-N₃ is SG3457:

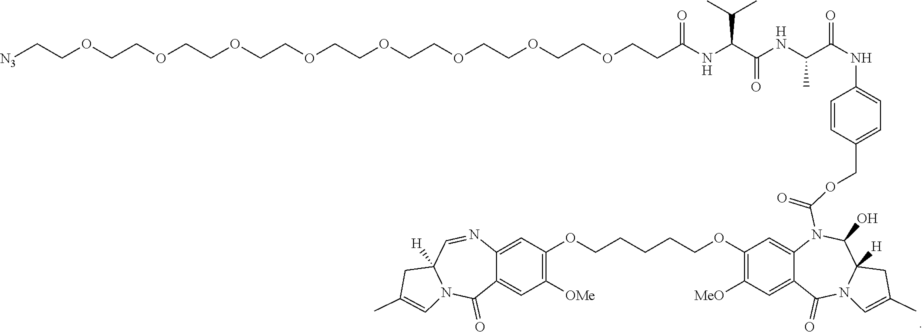 [SMILES: COc1cc2c(cc1OCCCCCOc1cc3c(cc1OC)C(=O)N1C=C(C)C[C@H]1[C@H](O)N3C(=O)OCc1ccc(NC(=O)[C@H](C)NC(=O)[C@@H](NC(=O)CCOCCOCCOCCOCCOCCOCCOCCOCCN=[N+]=[N-])C(C)C)cc1)N=C[C@@H]1CC(C)=CN1C2=O]

71. The conjuigate according to either embodiment 69 or embodiment 70, wherein DL-2-O—NH₂ is O-vc-PAB-MMAE:

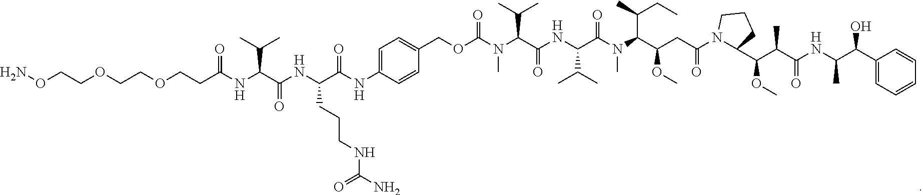 [SMILES: CC[C@H](C)[C@@H]([C@@H](CC(=O)N1CCC[C@@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCON)C(C)C)cc1)C(C)C]

72. The conjugate or mixture according to any one of embodiments 56 to 71, for use in therapy.

73. A pharmaceutical composition comprising the conjugate or mixture of any one of embodiments 56 to 63 and a pharmaceutically acceptable diluent, carrier or excipient.

74. The conjugate or mixture according to any one of embodiments 56 to 71, or the pharmaceutical composition according to embodiment 73, for use in the treatment of a proliferative disease in a subject.

75. The conjugate or mixture according to embodiment 74, wherein the disease is cancer.

76. Use of a conjugate or mixture according to any one of statements embodiments 56 to

71. or the pharmaceutical composition according to embodiment 73 in a method of medical treatment.

77. A method of medical treatment comprising administering to a patient the pharmaceutical composition of embodiment 73.

78. The method of embodiment 69 wherein the method of medical treatment is for treating cancer.

79. The method of embodiment 78, wherein the patient is administered a chemotherapeutic agent, in combination with the conjugate.

80. Use of a conjugate or mixture according to any one of embodiments 56 to 71 in a method of manufacture of a medicament for the treatment of a proliferative disease.

81. A method of treating a mammal having a proliferative disease, comprising administering an effective amount of conjugate or mixture according to any one of embodiments 56 to 71, or the pharmaceutical composition according to embodiment 73.

82. A drug-linker of one of the following formulae (IVa-1, IVa-2, IVb, IVc-1, IVc-2):

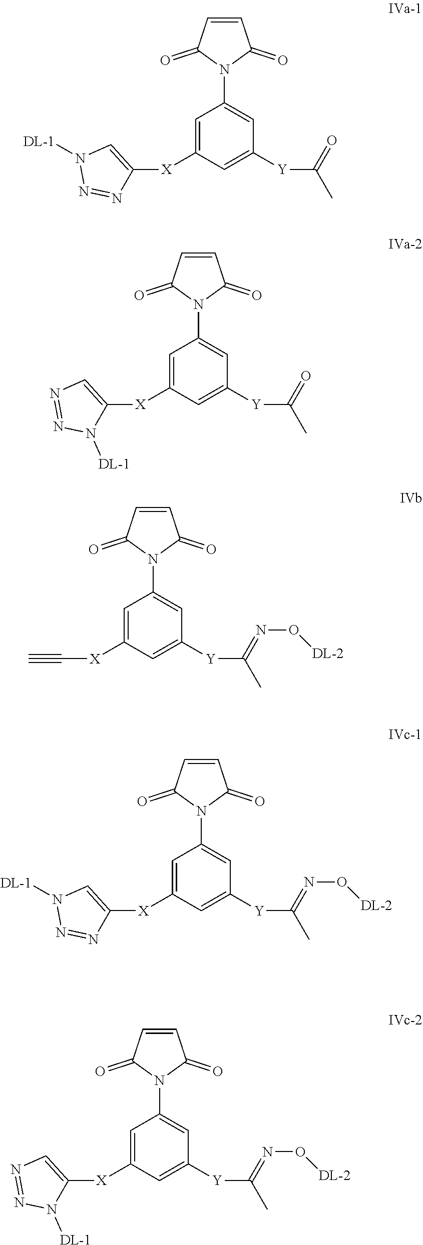

where X and Y are as defined in any one of embodiments 1 to 53, DL-1 and DL-2 are as defined in any one of embodiments 55 and 64 to 71.

83. A modified cell binding agent comprising a moiety of formula (V):

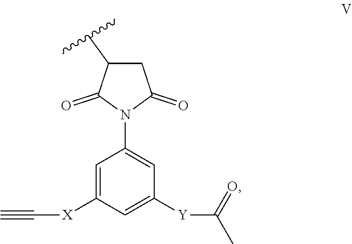

where X and Y are as defined in any one of embodiments 1 to 53.

1. A compound of formula I: 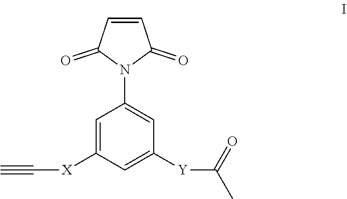
where:
X is —(C(═O)—NH)ₓₐ—(CH₂)_{xb}—(C₂H₄O)_{xc}—(CH₂)_{xd}^{≡}, where xa is 0 or 1, xb is 0-3, xc is 0 to 4 and xd is 0-3; and
Y is —(C(═O)-—NH)_{ya}—(CH₂)_{yb}—(C₂H₄O)_{yc}—(CH₂)_{yd}—(NH—C(═O))_{ye}—(CH₂)_{yf}—^{C═OMe}, where ya is 0 or 1, yb is 0-3, yc is 0 to 4, yd is 0-3, ye is 0 or 1, and yf is 0-3, with the provisos that:
(i) when xc is not 0, xd cannot be 0; and
(ii) when yc and ye are both not 0, yd cannot be 0.

2. A compound according to claim 1, wherein all of xa, xb, xc and xd are 0.

3. A compound according to claim 1, wherein xa is 0, xb is 0-3, xc is 1 to 4 and xd is 1-3.

4. A compound according to claim 1, wherein xa is 1, xb is 0, xc is 0 and xd is 1-3.

5. A compound according to claim 1, wherein all of ya, yb, yc, yd, ye and yf are 0.

6. A compound according to claim 1, wherein ye is 1, yb is 0, yc is 1 to 4, yd is 1-3, ye is 1 and yf is 1-3.

7. A compound according to claim 1, wherein X and Y are selected from:

| | X | Y |
|---|---|---|
| (i) | Single bond | Single bond |
| (ii) | 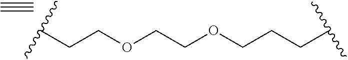 | Single bond |
| (iii) | 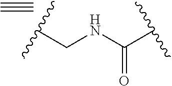 | 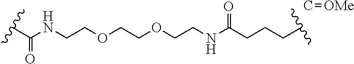 |

8. A linker between one or two payloads and a cell binding agent comprising one of the following moieties (IIa-1, IIa-2, IIb, IIc-1, IIc-2): 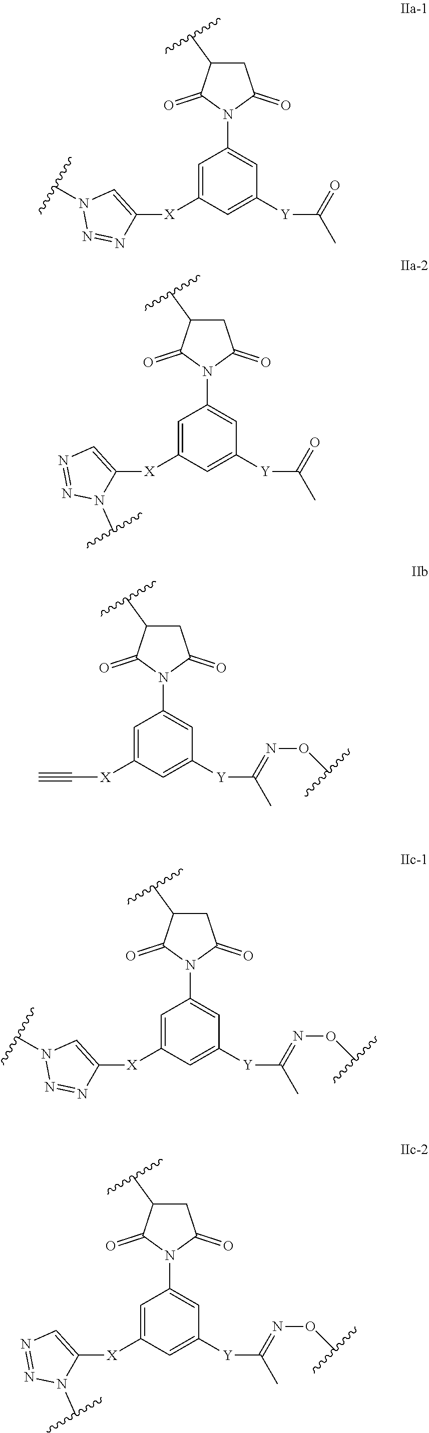
where:
X is —(C(═O)—NH)ₓₐ—(CH₂)_{xb}(C₂H₄O)_{xc}—(CH₂)_{xd}—^{≡}, where xa is 0 or 1, xb is 0-3, xc is 0 to 4 and xd is 0-3; and
Y is —(C(═O)—NH)_{ya}(C₂)_{yb}—(C₂H₄O)_{yc}—(CH₂)_{yd}—(NH—C(═O))_{ye}—(CH₂)_{yf}—^{C═OMe}, where ya is 0 or 1, yb is 0-3, yc is 0 to 4 yd is 0-3. ye is 0 or 1 and f is 0-3, with the provisos that:
(i) when xc is not 0, xd cannot be 0; and
(ii) when yc and ye are both not 0, yd cannot be 0.

9. A conjugate of one of the following formulae (IIIa-1, IIIa-2, IIIb, IIIc-1, IIIc-2): 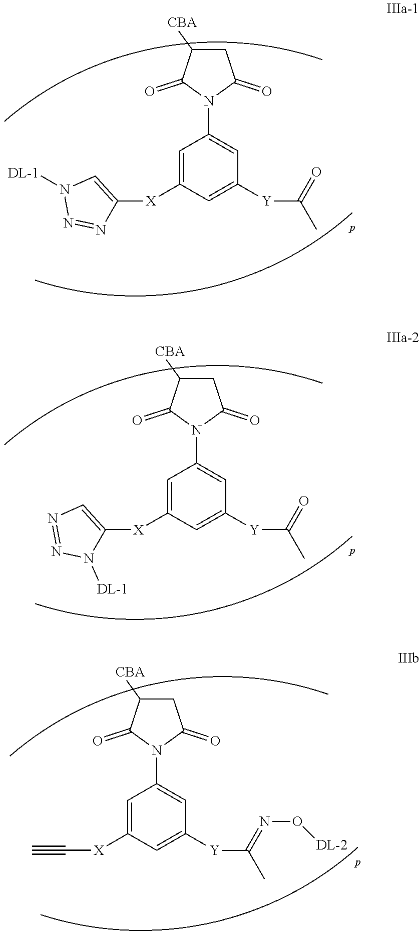 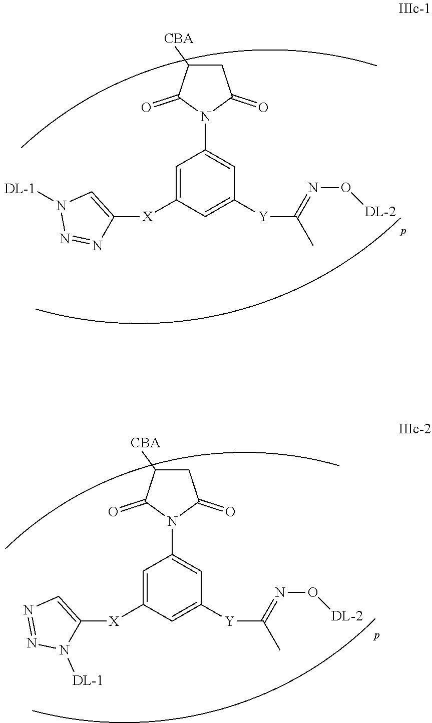
where:
X is —(C(═O)—NH)ₓₐ—(CH₂)_{xb}—(C₂H₄O)_{xc}—(CH₂)_{xd}—^{≡}, where xa is 0 or 1, xb is 0-3, xc is 0 to 4 and xd is 0-3; and
Y is —(C(═O)—NH)_{ya}—(CH₂)_{yb}—(C₂H₄O)_{yc}—(CH₂)_{yd}—(NH—C(═O))_{ye}—(CH₂)_{yf}—^{C═OMe}, where ya is 0 or 1, yb is 0-3, yc is 0 to 4 yd is 0-3. ye is 0 or 1 and f is 0-3, with the provisos that:
(i) when xc is not 0, xd cannot be 0; and
(ii) when yc and ye are both not 0, yd cannot be 0,
CBA is a cell binding agent, DL-1 is a first drug-linker moiety, DL-2 is a second drug-linker moiety, and p is from 1 to 10.

10. The conjugate according to claim 9, wherein the cell binding agent is an antibody or an active fragment thereof.

11. The conjugate according to claim 10, wherein the antibody or antibody fragment is an antibody or antibody fragment for a tumour-associated antigen.

12. The conjugate according to claim 10, wherein the antibody or antibody fragment is a cysteine-engineered antibody.

13. The conjugate according to claim 9, wherein drugs in the first drug-linker moiety and second drug-linker moiety (if present) are selected from the group consisting of cytotoxins, antiviral agents, antibacterials agents, peptides and oligonucleotides.

14. The conjugate according to claim 13, wherein a cytoxin is selected from the group consisting of colchicine, vinca alkaloids, anthracyclines, camptothecins, doxorubicin, daunorubicin, taxanes, calicheamycins, tubulysins, irinotecans, an inhibitory peptide, amanitin, deBouganin, duocarmycins, maytansines, pyrrolobenzodiazepines (including dimers thereof) and auristatins.

15. The conjugate according to claim 14, wherein DL-1-N₃ is SG3457:

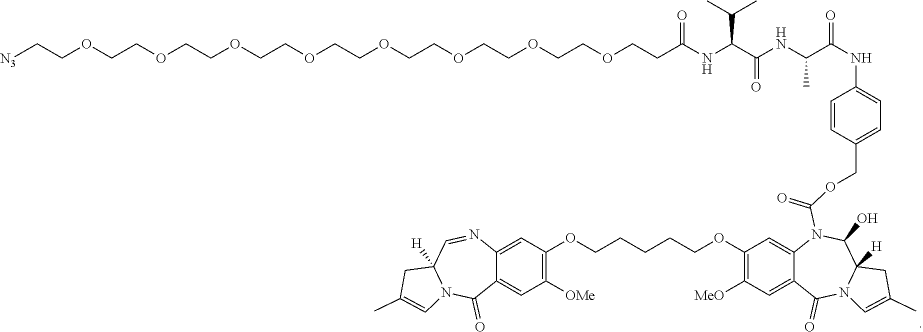 [SMILES: COc1cc2c(cc1OCCCCCOc1cc3c(cc1OC)C(=O)N1C=C(C)C[C@H]1[C@H](O)N3C(=O)OCc1ccc(NC(=O)[C@H](C)NC(=O)[C@@H](NC(=O)CCOCCOCCOCCOCCOCCOCCOCCOCCN=[N+]=[N-])C(C)C)cc1)N=C[C@@H]1CC(C)=CN1C2=O]

16. The conjuigate according to claim 14, wherein DL-2-O—NH₂ is O-vc-PAB-MMAE:

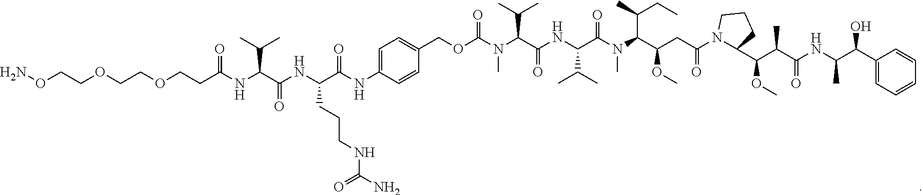 [SMILES: CC[C@H](C)[C@@H]([C@@H](CC(=O)N1CCC[C@@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCON)C(C)C)cc1)C(C)C]

17. A pharmaceutical composition comprising the conjugate of claim 9 and a pharmaceutically acceptable diluent, carrier or excipient.

18. (canceled).

19. A drug-linker of one of the following formulae (IVa-1, IVa-2, IVb, IVc-1, IVc-2): 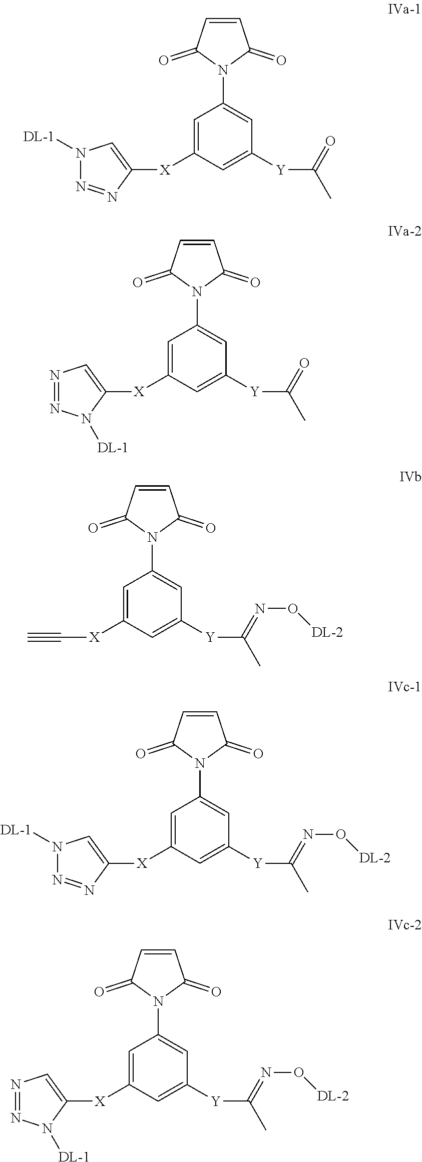
where:
X is —(C(═O)—NH)ₓₐ—(CH₂)_{xb}—(C₂H₄O)_{xc}—(CH₂)_{xd}—^{≡}, where xa is 0 or 1. xb is 0-3, xc is 0 to 4 and xd is 0-3; and
Y is —(C(═O)—NH)_{ya}—(CH₂)_{yb}—(C₂H₄O)_{yc}—(CH₂)_{yd}—(NH—C(═O))_{ye}—(CH₂)_{yf}—^{C═OMe}, where ya is 0 or 1, yb is 0-3 yc is 0 to 4, yd is 0-3, ye is 0 or 1, and yf is 0-3, with the provisos that:
(i) when xc is not 0, xd cannot be 0: and
(ii) when yc and ye are both not 0, yd cannot be 0.
DL-1 is a first drug-linker moiet and DL-2 is a second drug-linker moiety.

20. A modified cell binding agent comprising a moiety of formula (V): 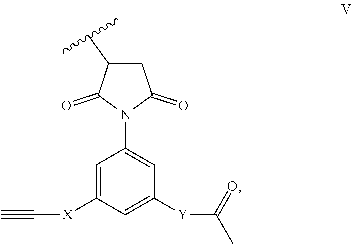
where:
X is —(C(═O)—NH)ₓₐ—(CH₂)_{xb}—(C₂H₄O)_{xc}—(CH₂)_{xd}—^{≡}, where xa is 0 or 1, xb is 0-3, xc is 0 to 4 and xd is 0-3; and
Y is —(C(═O)—NH)_{ya}—(CH₂)_{yb}—(C₂H₄O)_{yc}—(CH₂)_{yd}—(NH—C(═O))_{ye}—(CH₂)_{yf}—^{C═OMe}, where ya is 0 or 1, yb is 0-3, yc is 0 to 4 yd is 0-3, ye is 0 or 1 and yf is 0-3,
with the provisos that:
(i) when xc is not 0, xd cannot be 0; and
(ii) when yc and ye are both not 0, yd cannot be 0.
